(19) United States
(12) Patent Application Publication
(10) Pub. No.: US 2025/0061999 A1
(43) Pub. Date: Feb. 20, 2025

(54) TMS PROTOCOL AND ASSESSMENT

(71) Applicants: Mor Research Applications Ltd., Ramat Gan (IL); Ramot at Tel-Aviv University Ltd., Tel-Aviv (IL)

(72) Inventors: Amir GLIK, Ramat Gan (IL); Miriam FURST-YUST, Tel Aviv (IL); Felix BENNINGER, Ramat Gan (IL)

(73) Assignees: Mor Research Applications Ltd., Ramat Gan (IL); Ramot at Tel-Aviv University Ltd., Tel-Aviv (IL)

(21) Appl. No.: 18/719,323

(22) PCT Filed: Dec. 13, 2022
(86) PCT No.: PCT/IL2022/051318

§ 371 Date: Jun. 13, 2024

Related U.S. Application Data
(60) Provisional application No. 63/288,733, filed on Dec. 13, 2021.

(51) Int. Cl. G16H 20/40; A61N 2/00; A61N 2/02

(52) U.S. Cl. CPC G16H 20/40; A61N 2/006; A61N 2/02

(57) ABSTRACT

A system which can be used for carrying out a method for determining an efficacy of a transcranial magnetic stimulation (TMS) treatment, including:
delivering TMS to a patient using a TMS protocol in a TMS treatment session;
recording EEG signals before and after the delivering during the treatment session;
recording EEG signals after completion of the treatment session;
analyzing the recorded EEG signals to generate values of one or more EEG parameters;
determining an acute effect and an intermediate effect of the TMS protocol on a brain state of the patient based on a change in values of the one or more EEG parameters;
determining an efficacy of the TMS protocol based on the determined acute effect and the intermediate effect.

### RELATED APPLICATION/S

This application claims the benefit of priority under 35 USC § 119 (e) of U.S. Provisional Patent Application No. 63/288,733 filed 13 Dec. 2021, the contents of which are incorporated herein by reference in their entirety.

### FIELD AND BACKGROUND OF THE INVENTION

The present invention, in some embodiments thereof, relates to assessment of a treatment for neurological disorders and, more particularly, but not exclusively, to assessment of transcranial magnetic stimulation (TMS) treatment.

### SUMMARY OF THE INVENTION

The following describes some examples of embodiments of the invention (an embodiment may include features from more than one example and/or fewer than all features of an example): Example 1. A method for determining an efficacy of a transcranial magnetic stimulation (TMS) treatment, comprising:
delivering TMS to a patient using a TMS protocol in a TMS treatment session;
recording EEG signals before and after said delivering during said treatment session;
recording EEG signals after completion of said treatment session;
analyzing said recorded EEG signals to generate values of one or more EEG parameters;
determining an acute effect and an intermediate effect of said TMS protocol on a brain state of said patient based on a change in values of said one or more EEG parameters;
determining an efficacy of said TMS protocol based on said determined acute effect and said intermediate effect.

Example 2. A method according to example 1, wherein said recording said EEG signals after completion of said treatment session comprises recording said EEG signals after said patient sleeps.

Example 3. A method according to any one of examples 1 or 2, wherein said determining said efficacy comprises determining an ability of said TMS protocol to produce a desired result on a brain state and/or a cognitive state of said patient.

Example 4. A method according to any one of the previous examples, wherein said determining an efficacy comprises determining that said TMS protocol has a desired efficacy based on a determined acute effect which is a desired acute effect and a determined intermediate effect which is a desired intermediate effect, and wherein said method comprises repeating said delivering using said TMS protocol in an additional treatment session.

Example 5. A method according to example 4 comprising, extending an interval between said TMS treatment session and said additional treatment session of said TMS treatment based on said determined efficacy.

Example 6. A method according to any one of examples 1 to 3, wherein said determining an efficacy comprises determining that said TMS protocol has high efficacy immediately after completing said delivering and during the treatment session, and lower efficacy after a time period larger than 24 hours after the completion of said treatment session, and wherein said method comprises modifying said TMS treatment according to said determined efficacy.

Example 7. A method according to example 6, wherein said modifying comprises shortening an interval between two consecutive treatment sessions of said TMS treatment, according to said determined efficacy.

Example 8. A method according to any one of examples 6 or 7, wherein said modifying comprises replacing said TMS protocol with a different TMS protocol, or modifying values of at least one parameter of said TMS protocol, according to said determined efficacy.

Example 9. A method according to example 8, wherein said at least one parameter comprises at least one of, location of a magnetic coil on a head of said patient, intensity of a magnetic stimulation pulse, number of pulses per train of pulses, frequency of pulses during a train of pulses, and number of trains per treatment session.

Example 10. A method according to any one of examples 1 to 3, comprising:
determining that a responsiveness of a brain of said patient to said delivered TMS is lower than a desired responsiveness level and/or that the TMS treatment effect has reached a maximal therapeutic effect after said treatment session, based on said determined efficacy; and
modifying said TMS treatment according to said determined responsiveness and/or said determined TMS treatment effect.

Example 11. A method according to example 10, wherein said modifying comprises replacing said TMS protocol with a different TMS protocol, or modifying values of at least one parameter of said TMS protocol.

Example 12. A method according to example 11, wherein said at least one parameter comprises at least one of, location of a magnetic coil on a head of said patient, intensity of a magnetic stimulation pulse, number of pulses per train of pulses, frequency of pulses during a train of pulses, and number of trains per treatment session.

Example 13. A method according to any one of examples 10 to 12, wherein said modifying comprises extending an interval between two consecutive treatment sessions of said TMS treatment.

Example 14. A method according to any one of examples 10 to 13, wherein said modifying comprises administering at least one drug before and/or after said delivering.

Example 15. A method according to example 14, wherein said at least one drug comprises at least one of Donepezil, Rivastigmine, Remynil, Memantine, and Aducanumab.

Example 16. A method according to any one of examples 1 to 3, comprising performing a cognitive examination of said patient using at least one cognitive exam, after completing said treatment session, and evaluating a cognitive state of said patient based on the results of said cognitive examination, and wherein said determining comprises determining said efficacy of said TMS protocol based on said evaluated cognitive state.

Example 17. A method according to example 16, wherein said at least one cognitive exam comprises at least one of, a mini-mental state exam (MMSE), a Montreal Cognitive Assessment (MoCA) and Rey's Auditory Verbal Learning Test (RAVLT) exam.

Example 18. A method according to any one of examples 16 or 17, wherein said at least one cognitive exam comprises at least one of, Months backward test exam, Months forward test exam, Rey-Osterrieth Complex Figure Test (ROCFT) exam, Phonemic & Semantic Fluency exam, Digits span Forward & backwards exam, Stroop exam, Clock drawing exam, The Trail Making Test Part A and/or Part B, Functional Activities Questionnaire (FAQ), Quality of Life in Alzheimer's Disease (QOL-AD) Questionnaire, the Beck Depression Inventory (BDI, BDI-1A, BDI-II), the Beck Anxiety Inventory (BAI), and the Tablet-based Cognitive Assessment Tool (TabCAT).

Example 19. A method according to any one of examples 16 to 18, wherein said determined efficacy comprises determining that said TMS protocol has low efficacy in improving a cognitive state of said patient, and wherein said method further comprising modifying said TMS treatment according to said determined efficacy.

Example 20. A method according to example 19, wherein said modifying comprises replacing said TMS protocol with a different TMS protocol, or modifying values of at least one parameter of said TMS protocol, based on said determined low efficacy.

Example 21. A method according to example 20, wherein said at least one parameter comprises at least one of location of a magnetic coil on a head of said patient, intensity of a magnetic stimulation pulse, number of pulses per train of pulses, frequency of pulses during a train of pulses, and number of trains per treatment session.

Example 22. A method according to any one of examples 19 to 21, wherein said modifying comprises administering at least one drug before and/or after said delivering.

Example 23. A method according to example 22, wherein said at least one drug comprises at least one of Donepezil, Rivastigmine, Remynil, Memantine, and Aducanumab.

Example 24. A method according to any one of the previous examples, wherein said determining said intermediate effect comprises calculating a change in said one or more EEG parameters values generated from said EEG signals recorded after the completion of said treatment session and EEG signals recorded during the treatment session.

Example 25. A method according to any one of the previous examples, wherein said determining said acute effect comprises calculating a change in said one or more EEG parameter values generated from said EEG signals recorded before and after said delivering during said treatment session.

Example 26. A method according to any one of examples 1 to 3, wherein said delivering comprises delivering TMS to a patient in at least two treatment sessions, each using a different TMS protocol of at least two TMS protocols;
repeating said recording for each treatment session, and wherein said determining comprising determining an acute effect and an intermediate effect of each of said at least two TMS protocols, and wherein said determining an efficacy comprises determining an efficacy of each of said at least two TMS protocols based on said determined acute and intermediate effects.

Example 27. A method according to example 26, comprising selecting a TMS protocol of said at least two TMS protocols based on a determined efficacy of each of said at least two protocols.

Example 28. A method according to any one of the previous examples, wherein said one or more EEG parameters comprise one or more EEG microstates parameters, and wherein said analyzing comprises extracting said one or more EEG microstates parameters from said recorded EEG signals and measuring values of said one or more EEG microstates parameters or central tendency of said values, and wherein said acute effect and said intermediate effect are determined based on said measured values or said central tendency of said one or more EEG microstates parameters.

Example 29. A method according to example 28, wherein said measured one or more EEG microstates parameter comprise duration of at least one EEG microstate.

Example 30. A method according to any one of examples 28 or 29, wherein said measured one or more EEG microstates parameter comprise at least one of, frequency of at least one EEG microstate, coverage of at least one microstate and transitions between two or more EEG microstates.

Example 31. A method according to any one of the previous examples, wherein said delivering comprises delivering repetitive TMS to said patient.

Example 32. A method according to any one of the previous examples, wherein said delivering comprises delivering deep TMS to said patient.

Example 33. A method for determining an efficacy of a transcranial magnetic stimulation (TMS) treatment, comprising:
delivering TMS to a patient using a TMS protocol as part of a treatment session;
recording EEG signals before and after said delivering during said treatment session;
analyzing said recorded EEG signals to generate values of one or more EEG parameters;
determining an acute effect of said TMS protocol on a brain state of said patient based on values of said one or more EEG parameters;
performing a cognitive examination of said patient using at least one cognitive exam, after completing said treatment session to determine an intermediate effect of said TMS protocol, and
determining an efficacy of said TMS protocol based on said determined acute effect and said determined intermediate effect.

Example 34. A method according to example 33, wherein said at least one cognitive exam comprises at least one of, a mini-mental state exam (MMSE), a Montreal Cognitive Assessment (MoCA) and Rey's Auditory Verbal Learning Test (RAVLT) exam.

Example 35. A method according to any one of examples 33 or 34, wherein said at least one cognitive exam comprises at least one of, Months backward test exam, Months forward test exam, Rey-Osterrieth Complex Figure Test (ROCFT) exam, Phonemic & Semantic Fluency exam, Digits span Forward & backwards exam, Stroop exam, Clock drawing exam, The Trail Making Test Part A and/or Part B, Functional Activities Questionnaire (FAQ), Quality of Life in Alzheimer's Disease (QOL-AD) Questionnaire, the Beck Depression Inventory (BDI, BDI-1A, BDI-II), the Beck Anxiety Inventory (BAI), and the Tablet-based Cognitive Assessment Tool (TabCAT).

Example 36. A method according to any one of examples 33 to 35, wherein said determined efficacy comprises determining that said TMS protocol has low efficacy in improving a cognitive state of said patient, based on said intermediate effect, and wherein said method further comprising modifying said TMS treatment according to said determined efficacy.

Example 37. A method according to example 36, wherein said modifying comprises replacing said TMS protocol with a different TMS protocol, or modifying values of at least one parameter of said TMS protocol, based on said determined low efficacy.

Example 38. A method according to example 37, wherein said at least one parameter comprises at least one of location of a magnetic coil on a head of said patient, intensity of a magnetic stimulation pulse, number of pulses per train of pulses, frequency of pulses during a train of pulses, and number of trains per treatment session.

Example 39. A method according to any one of examples 36 to 38, wherein said modifying comprises administering at least one drug before and/or after said delivering.

Example 40. A method according to example 39, wherein said at least one drug comprises at least one of Donepezil, Rivastigmine, Remynil, Memantine, and Aducanumab.

Example 41. A method according to any one of examples 33 to 40, wherein said one or more EEG parameters comprise one or more EEG microstates parameters, and wherein said analyzing comprises extracting said one or more EEG microstates parameters from said recorded EEG signals and measuring values of said one or more EEG microstates parameters or central tendency of said values, and wherein said acute effect is determined based on said measured values of said one or more EEG microstates parameters or central tendency thereof.

Example 42. A method according to example 41, wherein said measured one or more EEG microstates parameter comprise duration of at least one EEG microstate Example 43. A method according to any one of examples 41 or 42, wherein said measured one or more EEG microstates parameter comprise at least one of, frequency of at least one EEG microstate, coverage of at least one microstate and transitions between two or more EEG microstates.

Example 44. A method for selecting a transcranial magnetic stimulation (TMS) protocol, comprising:
providing at least two TMS protocols;
delivering TMS to a patient in at least two consecutive TMS treatment sessions, each with a different TMS protocol of said at least two TMS protocols;
recording EEG signals before and after said delivering in each of said at least two consecutive TMS treatment sessions;
analyzing said recorded EEG signals to generate values of one or more EEG parameters;
determining an acute effect of each of said at least two TMS protocols on a brain state of said patient, based on a change in values of said one or more EEG parameters after said delivering in each treatment session;
selecting a TMS protocol of said at least two TMS protocols according to said determined acute effect of each of said at least two TMS protocols.

Example 45. A method according to example 44, wherein said selecting comprises selecting a TMS protocol of said at least two TMS protocols having a higher acute effect.

Example 46. A method according to any one of examples 44 or 45, wherein said one or more EEG parameters comprise one or more EEG microstates parameters, and wherein said analyzing comprises extracting said one or more EEG microstates parameters from said recorded EEG signals and measuring values of said one or more EEG microstates parameters or central tendency of said values.

Example 47. A method according to example 46, wherein said measured one or more EEG microstates parameter comprise duration of at least one EEG microstate.

Example 48. A method according to any one of examples 46 or 47, wherein said measured one or more EEG microstates parameter comprise at least one of, frequency of at least one EEG microstate, coverage of at least one microstate and transitions between two or more EEG microstates.

Example 49. A method for delivering a transcranial magnetic stimulation (TMS) treatment comprising:
positioning a magnetic coil of a TMS system on a scalp of a subject above a region of a prefrontal cortex;
delivering transcranial magnetic stimulation through said magnetic coil by delivering a series of pulses, wherein a power of each pulse of said series of pulses is adjusted according to a maximal tolerable power level of a subject, and wherein said series of pulses is delivered in a series of between 45-55 trains each containing between 18 and 22 pulses, wherein a duration of each train is between 1.8 and 2.2 seconds, and wherein an interval between two trains of pulses is between 18 and 22 seconds.

Example 50. A system for determining an efficacy of a transcranial magnetic stimulation (TMS) treatment protocol, comprising:
a control circuitry, wherein said control circuitry is configured to:
analyzing EEG signals recorded during a TMS treatment delivered to a patient, to generate values of one or more EEG parameters;
determining an acute effect and an intermediate effect of said TMS protocol on a brain state of said patient based on a change in values of said one or more EEG parameters; and
determine an efficacy of a TMS protocol used in said TMS treatment based on said determined acute effect and said determined intermediate effect.

Example 51. A system according to example 50, comprising:
a user interface operationally connected to said control circuitry, wherein said user interface is configured to generate a human detectable indication;
and wherein said control circuitry signals said user interface to generate said human detectable indication according to said determined efficacy.

Example 52. A system according to example 51, wherein said control circuitry signals said user interface to generate a human detectable indication with instructions to modify at least one parameter of said TMS protocol or to replace said TMS protocol with a different TMS protocol, if said determined efficacy is lower than a desired efficacy.

Example 53. A system according to example 51, wherein said control circuitry signals said user interface to generate a human detectable indication with instructions to stop said TMS treatment or to extend a length of an interval between two consecutive treatment sessions of said TMS treatment, if said determined efficacy is a desired efficacy or is larger than a desired efficacy.

Example 54. A system according to any one of examples 50 to 53, wherein said one or more EEG parameters comprise one or more EEG microstates parameters;
and wherein said control circuitry is configured to extract said one or more EEG microstates parameters from said EEG signals and to calculate values of said extracted one or more EEG parameters or central tendency thereof, during said analyzing, and to determine said acute effect and said intermediate effect based on a change in said calculated values of said extracted one or more EEG microstates parameters.

Example 55. A system according to example 54, wherein said one or more EEG microstates parameter comprise duration of at least one EEG microstate.

Example 56. A system according to any one of examples 54 or 55, wherein said one or more EEG microstates parameter comprises at least one of, frequency of at least one EEG microstate, coverage of at least one microstate and transitions between two or more EEG microstates.

Below are some additional examples of embodiments of the invention (an embodiment may include features from more than one example and/or fewer than all features of an example):

Example 1. A system for delivery of a transcranial magnetic stimulation (TMS) treatment to a patient, comprising:
at least one magnetic coil configured to be positioned close to, or in contact with a head of the patient;
at least one EEG electrode attached to a head of the patient, configured to record EEG signals;
a memory, wherein the memory stores at least one TMS treatment protocol;
a control circuitry, wherein the control circuitry is configured to:
deliver TMS to the patient using the at least one TMS protocol in at least one treatment session;
record EEG signals by the at least one electrode before and/or after the delivery of the TMS protocol during the treatment session;
record EEG signals by the at least one EEG electrode after completion of the treatment session;
analyze the recorded EEG signals to generate values of one or more EEG parameters;
determine an acute effect and an intermediate effect of the TMS protocol on a brain state of the patient based on a change in values of the one or more EEG parameters;
determine an efficacy of the TMS protocol based on the determined acute effect and the determined intermediate effect.

Example 2. A system according to example 1, wherein the recording the EEG signals by the control circuitry after completion of the treatment session comprises recording the EEG signals after the patient sleeps.

Example 3. A system, according to any one of the previous examples, wherein the determining the efficacy by the control circuitry comprises determining an ability of the TMS protocol to produce a desired result on a brain state and/or a cognitive state of the patient.

Example 4. A system according to any one of the previous examples, wherein the determining an efficacy by the control circuitry comprises determining that the TMS protocol has a desired efficacy based on a determined acute effect which is a desired acute effect and a determined intermediate effect which is a desired intermediate effect, and wherein the control circuitry is configured to deliver the TMS to the patient using the TMS protocol in an additional treatment session.

Example 5. A system according to example 4, wherein the control circuitry is configured to extend an interval between the TMS treatment session and the additional treatment session of the TMS treatment based on the determined efficacy.

Example 6. A system according to any one of examples 1 to 3, wherein the determining an efficacy by the control circuitry comprises determining that the TMS protocol has high efficacy immediately after completing the delivering and during the treatment session, and lower efficacy after a time period larger than 24 hours after the completion of the treatment session, and wherein the control circuitry is configured to modify the TMS treatment protocol according to the determined efficacy.

Example 7. A system according to example 6, wherein the control circuitry is configured to modify the stored TMS treatment protocol by shortening an interval between two consecutive treatment sessions of the TMS treatment protocol, according to the determined efficacy.

Example 8. A system according to any one of examples 6 or 7, wherein the control circuitry is configured to modify the stored TMS treatment protocol by replacing the stored TMS treatment protocol with a different TMS treatment protocol, or by modifying values of at least one parameter of the TMS treatment protocol, according to the determined efficacy.

Example 9. A system according to example 8, wherein the at least one parameter comprises at least one of, location of a magnetic coil on a head of the patient, intensity of a magnetic stimulation pulse, number of pulses per train of pulses, frequency of pulses during a train of pulses, and number of trains per treatment session.

Example 10. A system according to any one of examples 1 to 3, wherein the control circuitry is configured to:
determine that a responsiveness of a brain of the patient to the delivered TMS is lower than a desired responsiveness level and/or that an effect of the TMS treatment has reached a maximal therapeutic effect after the treatment session, based on the determined efficacy; and
modify the TMS treatment and/or the TMS treatment protocol according to the determined responsiveness and/or the determined TMS treatment effect.

Example 11. A system according to example 10, wherein the modify the TMS treatment protocol by the control circuitry comprises replacing the TMS protocol with a different TMS protocol, or modifying values of at least one parameter of the TMS protocol.

Example 12. A system according to example 11, wherein the at least one parameter comprises at least one of, location of a magnetic coil on a head of the patient, intensity of a magnetic stimulation pulse, number of pulses per train of pulses, frequency of pulses during a train of pulses, and number of trains per treatment session.

Example 13. A system according to any one of examples 10 to 12, wherein the modifying by the control circuitry comprises extending an interval between two consecutive treatment sessions of the TMS treatment.

Example 14. A system according to any one of examples 10 to 13, wherein the modifying by the control circuitry comprises generating an indication by the control circuitry to administer at least one drug before and/or after delivery of the TMS.

Example 15. A system according to any one of the previous examples, wherein the control circuitry is configured to generate an indication with instructions to perform a cognitive examination of the patient using at least one cognitive exam, after completing the treatment session, and evaluating a cognitive state of the patient based on the results of the cognitive examination, and wherein the control circuitry is configured to determine the efficacy of the stored TMS treatment protocol based on the evaluated cognitive state.

Example 16. A system according to example 15, wherein the at least one cognitive exam comprises at least one of, a mini-mental state exam (MMSE), a Montreal Cognitive Assessment (MoCA) and Rey's Auditory Verbal Learning Test (RAVLT) exam, Months backward test exam, Months forward test exam, Rey-Osterrieth Complex Figure Test (ROCFT) exam, Phonemic & Semantic Fluency exam, Digits span Forward & backwards exam, Stroop exam, Clock drawing exam, The Trail Making Test Part A and/or Part B, Functional Activities Questionnaire (FAQ), Quality of Life in Alzheimer's Disease (QOL-AD) Questionnaire, the Beck Depression Inventory (BDI, BDI-1A, BDI-II), the Beck Anxiety Inventory (BAI), and the Tablet-based Cognitive Assessment Tool (TabCAT).

Example 17. A system according to any one of examples 15 or 16, wherein the determined efficacy comprises determining that the stored TMS treatment protocol has low efficacy in improving a cognitive state of the patient, and wherein the control circuitry is configured to modify the TMS treatment according to the determined efficacy.

Example 18. A system according to example 17, wherein the control circuitry is configured to modify the TMS treatment by replacing the stored TMS treatment protocol with a different TMS treatment protocol, or by modifying values of at least one parameter of the stored TMS treatment protocol, based on the determined low efficacy.

Example 19. A system according to example 18, wherein the at least one parameter comprises at least one of location of a magnetic coil on a head of the patient, intensity of a magnetic stimulation pulse, number of pulses per train of pulses, frequency of pulses during a train of pulses, and number of trains per treatment session.

Example 20. A system according to any one of examples 17 to 19, wherein the control circuitry is configured to generate an indication with instructions to administer at least one drug before and/or after the delivering of the TMS treatment to the patient, according to the determined efficacy.

Example 21. A system according to any one of the previous examples, wherein the control circuitry is configured to determine the intermediate effect by calculating a change in the one or more EEG parameters values generated from the EEG signals recorded after the completion of the treatment session, and the one or more EEG parameters values generated from the EEG signals recorded during the treatment session.

Example 22. A system according to any one of the previous examples, wherein the control circuitry is configured to determine the acute effect by calculating a change in the one or more EEG parameter values generated from the EEG signals recorded before and after the delivering during the treatment session.

Example 23. A system according to any one of examples 1 to 3, wherein said at least one TMS treatment protocol comprises at least two TMS protocols, wherein the delivering by the control circuitry comprises delivering TMS to a patient in at least two treatment sessions, each using a different TMS protocol of said at least two TMS protocols stored in the memory; and wherein the control circuitry is configured to determine an efficacy of each of the at least two TMS protocols by repeating the recording and the determining, and based on the determined acute and intermediate effects.

Example 24. A system according to example 23, wherein the control circuitry is configured to select a TMS protocol of the at least two TMS treatment protocols based on a determined efficacy of each of the at least two protocols.

Example 25. A system according to any one of the previous examples, wherein the one or more EEG parameters comprise one or more EEG microstates parameters, and wherein the analyzing by the control circuitry comprises extracting the one or more EEG microstates parameters from the recorded EEG signals and measuring values of the one or more EEG microstates parameters or central tendency of the values, and wherein the acute effect and the intermediate effect are determined by the control circuitry based on the measured values or the central tendency of the one or more EEG microstates parameters.

Example 26. A system according to example 25, wherein the one or more EEG microstates parameter comprise duration of at least one EEG microstate.

Example 27. A system according to any one of examples 25 or 26, wherein the one or more EEG microstates parameter comprise at least one of, frequency of at least one EEG microstate, coverage of at least one microstate and transitions between two or more EEG microstates.

Example 28. A system according to any one of the previous examples, wherein the delivering of the TMS by the control circuitry comprises delivering repetitive TMS and/or deep TMS to the patient.

Example 29. A system for delivery of a transcranial magnetic stimulation (TMS) treatment to a patient, comprising:
at least one magnetic coil configured to be positioned close to, or in contact with a head of the patient;
at least one electrode attached to a head of the patient, configured to record EEG signals;
a memory, wherein the memory stores at least one TMS treatment protocol;
a control circuitry, wherein the control circuitry is configured to:
deliver TMS to the patient using the at least one TMS treatment protocol as part of a treatment session;
record EEG signals by the at least one electrode before and after the delivery of the TMS during the treatment session;
analyze the recorded EEG signals to generate values of one or more EEG parameters;
determine an acute effect of the at least one TMS treatment protocol on a brain state of the patient based on a change in values of the one or more EEG parameters;
generate an indication to perform a cognitive examination of the patient using at least one cognitive exam, after completing the treatment session, to determine an intermediate effect of the at least one TMS treatment protocol, and
determine an efficacy of the TMS protocol based on the determined acute effect and the determined intermediate effect.

Example 30. A system according to example 29, wherein the at least one cognitive exam comprises at least one of, a mini-mental state exam (MMSE), a Montreal Cognitive Assessment (MoCA) and Rey's Auditory Verbal Learning Test (RAVLT) exam, Months backward test exam, Months forward test exam, Rey-Osterrieth Complex Figure Test (ROCFT) exam, Phonemic & Semantic Fluency exam, Digits span Forward & backwards exam, Stroop exam, Clock drawing exam, The Trail Making Test Part A and/or Part B, Functional Activities Questionnaire (FAQ), Quality of Life in Alzheimer's Disease (QOL-AD) Questionnaire, the Beck Depression Inventory (BDI, BDI-1A, BDI-II), the Beck Anxiety Inventory (BAI), and the Tablet-based Cognitive Assessment Tool (TabCAT).

Example 31. A system according to any one of examples 29 or 30, wherein the determined efficacy comprises determining that the TMS protocol has low efficacy in improving a cognitive state of the patient, based on the intermediate effect, and wherein the control circuitry is configured to modify the TMS treatment according to the determined efficacy.

Example 32. A system according to example 31, wherein the modifying comprises replacing the TMS protocol with a different TMS protocol, or modifying values of at least one parameter of the TMS protocol, based on the determined low efficacy.

Example 33. A system according to example 32, wherein the at least one parameter comprises at least one of location of a magnetic coil on a head of the patient, intensity of a magnetic stimulation pulse, number of pulses per train of pulses, frequency of pulses during a train of pulses, and number of trains per treatment session.

Example 34. A system according to any one of examples 31 to 33, wherein the modifying the treatment by the control circuitry comprises generating an indication by the control circuitry with instructions to administer at least one drug before and/or after the delivering of the TMS by the control circuitry.

Example 35. A system according to any one of examples 29 to 34, wherein the one or more EEG parameters comprise one or more EEG microstates parameters, and wherein the analyzing by the control circuitry comprises extracting the one or more EEG microstates parameters from the recorded EEG signals and measuring values of the one or more EEG microstates parameters or central tendency of the values, and wherein the acute effect is determined by the control circuitry based on the measured values of the one or more EEG microstates parameters or central tendency thereof.

Example 36. A system according to example 35, wherein the measured one or more EEG microstates parameter comprise duration of at least one EEG microstate.

Example 37. A system according to any one of examples 35 or 36, wherein the measured one or more EEG microstates parameter comprise at least one of, frequency of at least one EEG microstate, coverage of at least one microstate and transitions between two or more EEG microstates.

Example 38. A system for determining an efficacy of a transcranial magnetic stimulation (TMS) treatment protocol, comprising:
a control circuitry, wherein the control circuitry is configured to:
analyzing EEG signals recorded during a TMS treatment delivered to a patient, to generate values of one or more EEG parameters;
determining an acute effect and an intermediate effect of the TMS protocol on a brain state of the patient based on a change in values of the one or more EEG parameters; and
determine an efficacy of a TMS protocol used in the TMS treatment based on the determined acute effect and the determined intermediate effect.

Example 39. A system according to example 38, comprising:
a user interface operationally connected to the control circuitry, wherein the user interface is configured to generate a human detectable indication;
and wherein the control circuitry signals the user interface to generate the human detectable indication according to the determined efficacy.

Example 40. A system according to example 39, wherein the control circuitry signals the user interface to generate a human detectable indication with instructions to modify at least one parameter of the TMS protocol or to replace the TMS protocol with a different TMS protocol, if the determined efficacy is lower than a desired efficacy.

Example 41. A system according to example 40, wherein the control circuitry signals the user interface to generate a human detectable indication with instructions to stop the TMS treatment or to extend a length of an interval between two consecutive treatment sessions of the TMS treatment, if the determined efficacy is a desired efficacy or is larger than a desired efficacy.

Example 42. A system according to any one of examples 38 to 41, wherein the one or more EEG parameters comprise one or more EEG microstates parameters;
and wherein the control circuitry is configured to extract the one or more EEG microstates parameters from the EEG signals and to calculate values of the extracted one or more EEG parameters or central tendency thereof, during the analyzing, and to determine the acute effect and the intermediate effect based on a change in the calculated values of the extracted one or more EEG microstates parameters.

Example 43. A system according to example 42, wherein the one or more EEG microstates parameter comprise at least one of, duration of at least one EEG microstate, frequency of at least one EEG microstate, coverage of at least one microstate and transitions between two or more EEG microstates.

Example 44. A system for delivery of a transcranial magnetic stimulation (TMS) treatment to a patient, comprising:
at least one magnetic coil configured to be positioned close to, or in contact with a head of the patient;
a control circuitry, wherein the control circuitry is configured to signal the at least one magnetic coil to generate and deliver a series of pulses, wherein a power of each pulse of the series of pulses is adjusted according to a maximal tolerable power level of a subject, and wherein the series of pulses is delivered in a series of between 45-55 trains each containing between 18 and 22 pulses, wherein a duration of each train is between 1.8 and 2.2 seconds, and wherein an interval between two trains of pulses is between 18 and 22 seconds.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a flow chart of a general process for determining an efficacy of a TMS protocol or a TMS treatment, according to some exemplary embodiments of the invention;

FIG. 1B is a flow chart of a general process for evaluating a TMS treatment, according to some exemplary embodiments of the invention;

FIGS. 2A and 2B are flow charts of a detailed process for evaluating a TMS treatment, according to some exemplary embodiments of the invention;

FIG. 3 is a flow chart of a process for selecting a TMS protocol, according to some exemplary embodiments of the invention;

FIG. 4 is a block diagram of a device for microstates measurements, according to some exemplary embodiments of the invention;

FIG. 5 is a flow chart of a process for personalizing a TMS protocol, according to some exemplary embodiments of the invention;

FIG. 6A is a flow chart of a general process for extracting and analyzing microstates, according to some embodiments of the invention;

FIG. 6B is a detailed flow chart for extraction of microstates parameters according to some embodiments of the invention;

FIG. 7 is a flow chart of a process for extraction of EEG quiet segments, according to some embodiments of the invention;

FIG. 8 is a flow chart of a process for extraction of microstates from an EEG signal, according to some embodiments of the invention;

FIG. 9 is a flow chart of a process for calculating duration of one or more microstates, according to some embodiments of the invention;

FIG. 10A is a graph showing a change in microstates mean duration between different treatment sessions of a TMS treatment experiment;

FIG. 10B is a bar graph showing changes in coverage percentage of each microstate before and after at least one treatment session of the TMS treatment experiment;

FIG. 10C is a dot graph showing a relative change in microstate duration for each microstate and for each treatment session, divided per TMS protocol used in the treatment sessions of the TMS treatment experiment;

FIGS. 10D-10I are graphs showing additional examples of analysis of microstates parameters as performed during treatment session of the TMS treatment experiment;

FIG. 11A is a graph showing a correlation between changes in microstates mean duration and changes in mini-mental test scores during the TMS treatment experiment; and

FIGS. 11B-11P are additional examples of graphs showing correlation between changes in microstates mean duration and scores of additional cognitive exams performed during the TMS treatment experiment for a specific patient annotated as AA;

FIGS. 12A-12P are additional examples of graphs showing correlation between changes in microstates mean duration and scores of additional cognitive exams performed during the TMS treatment experiment for a specific patient annotated as RS; and

FIGS. 13A-13P are additional examples of graphs showing correlation between changes in microstates mean duration and scores of additional cognitive exams performed during the TMS treatment experiment for a specific patient annotated as YG.

### DESCRIPTION OF SPECIFIC EMBODIMENTS OF THE INVENTION

The present invention, in some embodiments thereof, relates to assessment of a treatment for neurological disorders and, more particularly, but not exclusively, to assessment of transcranial magnetic stimulation (TMS) treatment.

An aspect of some embodiments of the invention relates to assessment of a TMS treatment by determining a short-term effect, and a long-term effect, for example an intermediate effect, based on microstates measurements, for example measurements of at least one microstates parameter. In some embodiments, the short term effect is determined based on microstates measurement performed up to 12 hours, for example up to 10 hours, up to 6 hours, up to 3 hours or any intermediate, smaller or larger time period following the TMS treatment. In some embodiments, the long-term effect is determined based on microstates measurements performed at least 24 hours following the TMS treatment, for example microstates measurements performed at least 48 hours, at least 96 hours, at least 1 week, following the TMS treatment. In some embodiments, the at least one microstates parameter comprises duration of one or more microstate, and the short and long term effects are determined based on a change in duration of one or more microstate following the treatment.

According to some embodiments, the short term effect of the TMS protocol is determined based on a difference in microstates measurements, for example a change in microstates duration, between microstates measured following the TMS treatment, and microstates measured prior to the TMS treatment, as measured in a single treatment session. In some embodiments, the long term effect is determined based on a difference in microstates measurements, between microstates measured in a first treatment session and microstates measured in a following second treatment session. Optionally, the long term effect is determined based on a difference between microstates measured at a beginning of the second treatment session and microstates measured at the end of the first treatment session. In some embodiments, each treatment session lasts less than 6 hours, for example less than 4 hours, less than 2 hours or any intermediate, smaller or larger time period.

According to some exemplary embodiments, the long term effect of the TMS treatment is determined based on microstates measurements and results of cognitive evaluation, for example using one or more cognitive exams. In some embodiments, the long term effect of the TMS treatment is determined based on a change in score of the one or more cognitive exams between a cognitive exam score calculated prior to the TMS treatment, for example a baseline cognitive exam score, and a cognitive exam score calculated following the treatment. In some embodiments, the one or more cognitive exams comprise at least one of, Mini-Mental State Examination (MMSE), Montreal Cognitive Assessment (MoCA) exam, Months backward test exam, Months forward test exam, Rey-Osterrieth Complex Figure Test (ROCFT) exam, Rey's Auditory Verbal Learning Test (RAVLT) exam, Phonemic & Semantic Fluency exam, Digits span Forward & backwards exam, Stroop exam, Clock drawing exam, the Trail Making Test Part A and/or Part B, Functional Activities Questionnaire (FAQ), Quality of Life in Alzheimer's Disease (QOL-AD) Questionnaire, the Beck Depression Inventory (BDI, BDI-1A, BDI-II), The Beck Anxiety Inventory (BAI), and The Tablet-based Cognitive Assessment Tool (TabCAT). In some embodiments, the one or more cognitive exams comprise at least one of the MMSE, MoCA and RAVLAT exams, that showed the highest correlation with microstate measurements.

According to some embodiments, a brain responsiveness to receive an additional TMS treatment session is evaluated based on the determined long term-effect. In some embodiments, the brain responsiveness is evaluated by monitoring an overall change in microstates measurements, for example a change in duration of at least one microstate, at the end of two or more consecutive treatment sessions. In some embodiments, an overall change in microstates measurements, indicating a beneficial effect of the TMS treatment, which decreases or remains stable, for example with less than 10%, less than 5%. Less than 2% change or any intermediate, smaller or larger percentage value of change between the two or more consecutive treatments, indicates a reduction or no responsiveness of the brain to the TMS treatment, for example to a specific TMS protocol used for the TMS treatment.

According to some embodiments, if the assessment of the TMS treatment indicates a reduction in a positive effect or no positive effect following the TMS treatment, based on the determined short term effect and the determined long-term effect, the TMS treatment is stopped. Alternatively, a protocol of the TMS treatment is modified or replaced. Alternatively or additionally, the TMS treatment is combined with an additional treatment, for example a pharmaceutical treatment. In some embodiments, the pharmaceutical treatment include administration of at least one drug comprising at least one of, Donepezil, Rivastigmine, Remynil, Memantine, and Aducanumab.

An aspect of some embodiments of the invention relates to assessment of a TMS treatment based on microstates measurements, for example measurements of at least one microstates parameter, and results of at least one cognitive exam. In some embodiments, the microstates measurements are used to determine a short and/or long-term effects of the TMS treatment. In some embodiments, the at least one cognitive exam is used to evaluate an effect of the TMS treatment on at least one domain of cognitive functioning. In some embodiments, the at least one cognitive exam is performed at least 12 hours, for example at least 24 hours, at least 1 week, at least one month, at least 6 months or any intermediate, shorter or longer time period after completing at least one TMS treatment session.

According to some embodiments, a TMS protocol used for the TMS treatment is selected in order to improve at least one domain of cognitive function of a subject. In some embodiments, the at least one cognitive exam is used to evaluate an improvement in the at least one cognitive domain following the TMS treatment. In some embodiments, the at least one cognitive function domain comprises at least one of, a sensation domain, for example multisensory cognitive function; a perception domain, for example object recognition and/or organizational strategies; motor skills and construction domains, for example copying, drawing, other praxic skills; attention and concentration domains, for example selective attention, sustained attention and/or vigilance; memory domains, for example working memory (verbal, spatial, object, location), working memory components (central executive, maintenance, manipulation), episodic/declarative memory (verbal, non-verbal (encoding/storage/retrieval/free recall, cued recall, forced-choice recognition), procedural memory, semantic memory, prospective memory (time-based, event-based)); executive function domains, for example reasoning, problem solving, component skill management; processing speed domains, for example semantically relevant, coding and tracking; language/verbal skills domains, for example naming, fluency, and reading and comprehension.

According to some embodiments, if the at least one domain of cognitive function does not improve following the TMS treatment or the improvement is not a desired improvement, the TMS protocol is modified or adjusted.

According to some embodiments, the microstates measurement are used to determine a timing of performing a cognitive task, for example by defining an expected improvement window based on the determined short and/or long term effects.

An aspect of some embodiments of the invention relates to delivering TMS in at least two consecutive treatment sessions, each with a different TMS protocol, and selecting the TMS protocol showing better effect, based on microstates measurements. In some embodiments, the microstates measurements are performed in each treatment session, before and after the delivery of the TMS treatment. In some embodiments, the microstates measurements are used to determine a short term effect of each TMS protocol by determining a relation between microstates measurements performed before and after TMS delivery in each TMS treatment session. Additionally or alternatively, the microstates measurements are used to determine a long-term effect of the TMS protocol by determining a relation between microstate measurements performed in a second TMS treatment session compared to microstates parameters measured in a previous TMS treatment session.

According to some embodiments, the microstates measurements comprise measurements of duration of at least one microstate. In some embodiments, the short term effect of each TMS protocol is determined based on a change in microstates measurements, for example reduction in duration of the at least one microstate, following the TMS treatment. In some embodiments, the long-term effect is determined based on a change between microstates measurements performed at the end of a first TMS treatment session when a first TMS protocol was used, and microstates measurements performed before and/or after the second TMS treatment session when a second TMS protocol is used.

According to some embodiments, a TMS protocol showing a larger change in microstates measurement in a desired direction is selected, based on the determined short-term effect of each TMS protocol. Alternatively or additionally, a TMS protocol showing a larger change in microstates measurement in a desired direction, is selected based on the determined long-term effect of each TMS protocol.

According to some embodiments, microstates are measured during a treatment session when the patient is at a clinic, for example before and/or after the delivery of a TMS treatment. Alternatively or additionally, microstates are measured between treatment sessions, for example when the patient is outside the clinic and/or at home.

According to some exemplary embodiments, the TMS treatment described herein is used to treat subjects, for example human subjects, and/or patients diagnosed with dementia, for example Alzheimer's disease (AD), vascular dementia, Lewy body dementia, Frontotemporal dementia and/or mixed dementia. In some embodiments, the TMS treatment described herein is used to treat human subjects, and/or patients diagnosed with mild cognitive impairment (MCI), or with an early stage of dementia.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary General Process for Determining an Efficacy of a TMS Protocol

According to some exemplary embodiments, a TMS treatment is delivered to a patient as a series, for example at least two, treatment sessions. In some embodiments, in each treatment session, optionally performed in a clinic, for example in a treatment room in the clinic, TMS is delivered to the patient. In some embodiments, after the completion of the treatment session, the patient is released from the clinic. In some embodiments, the efficacy of the delivered TMS treatment is monitored using EEG signals recorded during each treatment session, for example before and after the delivery of TMS, and EEG signals recorded after completing the treatment session. In some embodiments, EEG signals recorded after completing the treatment session are recorded after the patient sleeps for at least 1 hour, for example at least 3 hours, at least 5 hours or any intermediate, smaller or larger time period. Optionally, the EEG signals recorded after the completion of the treatment session are recorded after the patient sleeps a rapid eye movement (REM) sleep.

According to some exemplary embodiments, the recorded EEG signals are used to determine a short effect, for example an acute effect, and an intermediate effect, of the TMS protocol used in the treatment session. In some embodiments, the efficacy of the TMS protocol and/or the TMS treatment are determined based on the determined acute and intermediate effects, and is used in order to decide how to proceed with the TMS treatment delivered to the patient. A potential advantage of using EEG signals to monitor an efficacy of the TMS protocol or treatment may be that the EEG signals are used as an objective marker for the efficacy and can be easily and safely acquired with relatively small effort from the patient.

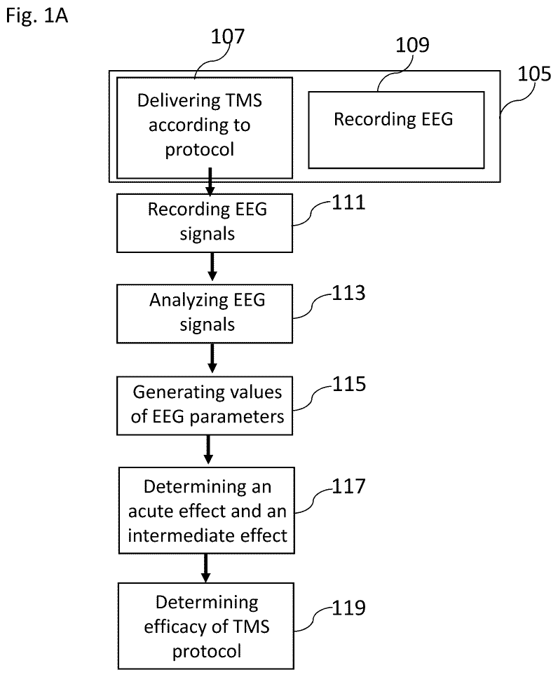

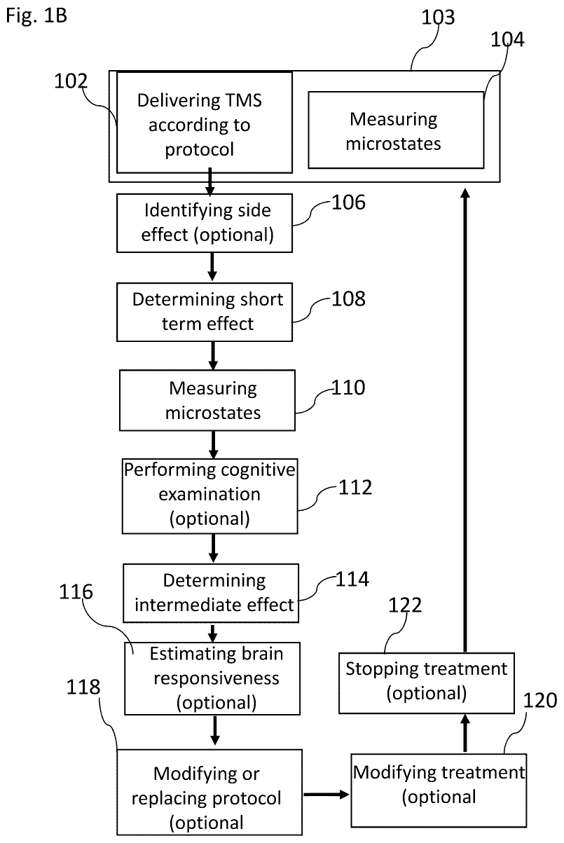

Reference is now made to FIG. 1A, depicting a process for determining an efficacy of a TMS protocol using EEG signals, according to some exemplary embodiments of the invention. In some embodiments, the method described in FIG. 1A, and other methods described in this application for determining an efficacy of a TMS protocol can also be used for determining an efficacy of a TMS treatment.

According to some exemplary embodiments, TMS is delivered to a patient using a TMS protocol, at block 107. In some embodiments, the TMS is delivered in a treatment session 105. In some embodiments, the TMS is delivered as repetitive TMS (rTMS). Optionally, the delivered TMS is deep TMS, for example a TMS that reaches a depth of up to 4 cm into the brain from the inner surface of skull of the patient. In some embodiments, the TMS protocol comprises one or more parameters, for example location of a magnetic coil on a head of the patient, intensity of a magnetic stimulation pulse, number of pulses per train of pulses, frequency of pulses during a train of pulses, and number of trains per treatment session.

According to some exemplary embodiments, EEG signals are recorded after the delivering of the TMS, during the treatment session. Optionally, the EEG signals are recorded immediately, for example up to 60 minutes, after 30 minutes or any intermediate, smaller or larger time period after the delivering at block 107 and during the treatment session 105. Additionally or optionally, EEG signals are also recorded prior to the delivering of the TMS, for example during the treatment session. In some embodiments, the EEG signals are recorded by at least one electrode attached to the patient head during the treatment session 105.

According to some exemplary embodiments, additional EEG signals are recorded after the completion of the treatment session, at block 111. In some embodiments, the additional EEG signals are recorded after the patient sleeps, for example REM sleeps, optionally for at least 1 hour, at least 2 hours, at least 5 hours or any intermediate, shorter or longer time period. In some embodiments, the additional EEG signals are recorded when the patient is at his home, or at the beginning of a following treatment session.

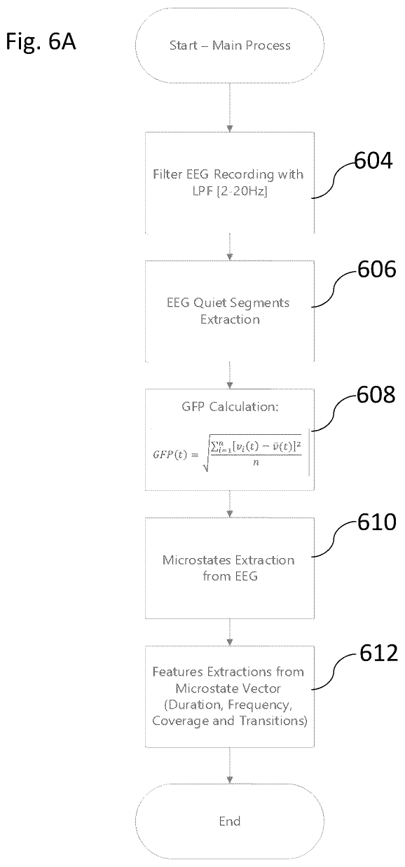

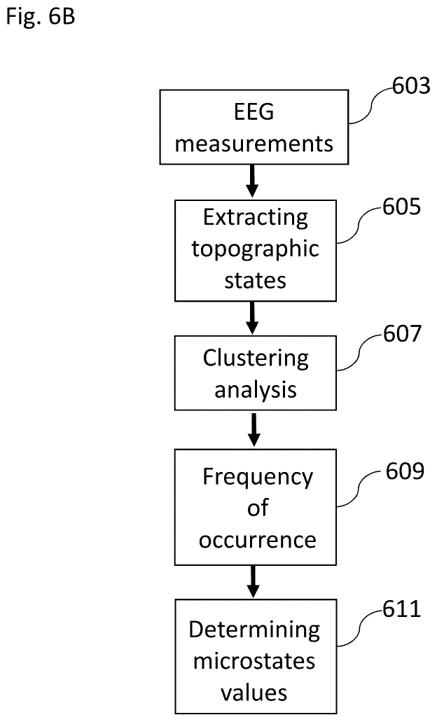

According to some exemplary embodiments, the EEG signals recorded at block 109 and 111 are analyzed, at block 113. In some embodiments, the analysis of the EEG signals comprises extracting of one or more parameters of the EEG signals. In some embodiments, the one or more EEG parameters comprise one or more microstate parameters, for example one or more microstate features. In some embodiments, the one or more EEG parameters comprise duration of at least one EEG microstate, frequency of at least one EEG microstate, coverage of at least one microstate and transitions between two or more EEG microstates, or measures of a central tendency thereof, for example arithmetic mean, median, and/or the mode. In some embodiments, the extraction of the one or more microstate parameters is described for example, in one or more of FIGS. 6A, 6B and 7-9.

According to some exemplary embodiments, values of the EEG parameters are generated at block 115. In some embodiments, values of the extracted microstates one or more parameters are generated at block 115. In some embodiments, the values are generated, for example calculated using one or more of the methods described in FIGS. 6A, 6B and 7-9.

According to some exemplary embodiments, an acute effect and an intermediate effect are determined at block 117. In some embodiments, the acute effect and the intermediate effect of the TMS protocol used in treatment session 105 are determined based on a change in the calculated values of the one or more microstates parameter. In some embodiments, the acute effect of the TMS protocol is determined based on a change in microstates parameter values between values calculated using EEG signals recorded prior to TMS delivering and values calculated using EEG signals recorded after the delivering at block 107 and during the treatment session. In some embodiments, the intermediate effect of the TMS protocol is determined based on a change in microstates parameter values between values calculated using EEG signals recorded after the completion of the treatment session 105, at block 111, and values calculated using EEG signals recorded during the treatment session, for example at block 109.

According to some exemplary embodiments, an efficacy of the TMS protocol is determined, at block 119. In some embodiments, the efficacy of the TMS protocol is an ability of the TMS protocol to produce a desired result on a brain state and/or a cognitive state of the patient. In some embodiments, the efficacy of the TMS protocol is determined based on the determined acute effect and the determined intermediate effect of the TMS protocol.

According to some exemplary embodiments, if the determined efficacy of the TMS protocol is a desired efficacy, for example a target efficacy, or higher than a desired efficacy, the TMS protocol is used in the following treatment session, for example in at least one additional treatment session of the TMS treatment. Alternatively or additionally, an interval, for example a time duration, between the treatment session 105 and a following treatment session is extended.

According to some exemplary embodiments, the determined efficacy of the TMS protocol reveals that the TMS protocol has high efficacy relative to a desired efficacy immediately after completing the delivering at block 107 during the treatment session 105 and low efficacy relative to a desired efficacy after the completion of the treatment session 105, for example at least 10 hours, at least 12 hours, at least 24 hours or any intermediate, smaller or larger time duration after the completion of the treatment session 105. In some embodiments, the TMS treatment is modified, for example by shortening an interval between two consecutive treatment sessions and/or by administering at least one drug before and/or after each TMS treatment session. Alternatively, values of at least one TMS protocol parameter are modified. In some embodiments, the at least one TMS protocol parameter comprises at least one of, location of a magnetic coil on a head of said patient, intensity of a magnetic stimulation pulse, number of pulses per train of pulses, frequency of pulses during a train of pulses, and number of trains per treatment session. Alternatively, the TMS protocol is replaced with a different TMS protocol.

According to some exemplary embodiments, a responsiveness of the patient brain for additional TMS delivery is determined based on the determined efficacy of the TMS protocol, for example when the TMS protocol efficacy is reduced, for example continuously reduced after two or more treatment sessions. Alternatively or additionally, the TMS protocol efficacy is used for example to determine whether the TMS treatment has reached a maximal therapeutic effect after at least one, or after two or more treatment sessions. In some embodiments, if the brain responsiveness for additional TMS treatment sessions, and/or if the maximal therapeutic effect has reached, the TMS treatment is modified. In some embodiments, the TMS treatment is modified, for example, by replacing the TMS protocol with a different TMS protocol. Alternatively, the TMS treatment is modified by modifying values of at least one treatment parameter, for example by modifying at least one of, location of a magnetic coil on a head of said patient, intensity of a magnetic stimulation pulse, number of pulses per train of pulses, frequency of pulses during a train of pulses, and number of trains per treatment session. Alternatively, the TMS treatment is modified by administering at least one drug before and/or after a TMS treatment session, for example Donepezil, Rivastigmine, Remynil, Memantine, and Aducanumab.

According to some exemplary embodiments, a cognitive examination using at least one cognitive exam is performed after the completion of the treatment session 105. Optionally, the cognitive examination is performed also prior to the treatment session 105 or to the delivery 107, for example to establish a baseline or a reference to following cognitive examinations. In some embodiments, the efficacy of the TMS protocol is determined based on the results of the cognitive examination in addition to the determined acute effects and the intermediate effects. In some embodiments, the at least one cognitive exam comprises at least one of, a mini-mental state exam (MMSE), a Montreal Cognitive Assessment (MoCA) and Rey's Auditory Verbal Learning Test (RAVLT) exam. Additionally, at least one cognitive exam comprises at least one of, Months backward test exam, Months forward test exam, Rey-Osterrieth Complex Figure Test (ROCFT) exam, Phonemic & Semantic Fluency exam, Digits span Forward & backwards exam, Stroop exam, Clock drawing exam, The Trail Making Test Part A and/or Part B, Functional Activities Questionnaire (FAQ),), Quality of Life in Alzheimer's Disease (QOL-AD) Questionnaire, the Beck Depression Inventory (BDI, BDI-1A, BDI-II), the Beck Anxiety Inventory (BAI), and the Tablet-based Cognitive Assessment Tool (TabCAT).

### Exemplary General Process for Evaluating TMS Treatment

According to some exemplary embodiments, a treatment for a neurological disorder, for example a transcranial magnetic stimulation (TMS) treatment is provided to a subject, for example a patient diagnosed with the neurological disorder. In some embodiments, the neurological disorder comprises at least one of addictions, schizophrenia, post-traumatic stress disorder (PTSD), Parkinson's, multiple sclerosis, chronic pain, post-stroke rehabilitation, bipolar, autism, alzheimer's/dementia, eating disorders, depression, postpartum depression, and tinnitus. In some embodiments, the TMS treatment is delivered using at least one TMS treatment protocol. In some embodiments, an effect of the TMS treatment when using a specific TMS treatment protocol, for example a short-term effect and an intermediate-term effect of the TMS treatment, is evaluated. In some embodiments, the effect is evaluated based on microstates measurements. Additionally or optionally, the effect is evaluated based on results of cognitive exams. Reference is now made to FIG. 1B, depicting a general process for evaluating a TMS treatment.

According to some exemplary embodiments, a TMS treatment is delivered to a patient using at least one TMS treatment protocol, at block 102. In some embodiments, the treatment is delivered during a treatment session 103. In some embodiments, the treatment is delivered according to values of one or more parameters of the protocol, for example treatment location (location of magnetic coil), pulse duration, pulse intensity (power), pulse frequency, number of pulses in a train of pulses, number of trains of pulses in a single treatment session, number of treatment sessions in a TMS treatment, and/or interval between two consecutive treatment sessions.

According to some exemplary embodiments, microstates are measured at block 104. In some embodiments, microstates are measured using signals, for example EEG signals, recorded from a subject brain. In some embodiments, the microstates are measured for example as described in International Patent Application Publication Number WO/2018/060878, filed on 27 Sep. 2017. In some embodiments, at least one parameter of the microstates, for example duration of each microstate, or duration of at least one microstate is measured at block 104. In some embodiments, the at least one microstates parameter comprises duration or mean duration of one or more microstates. Alternatively or additionally, the at least one microstates parameter comprises occurrence, frequency and/or number of transitions of one or more microstates.

According to some exemplary embodiments, the microstates are measured before and after the delivery of the TMS treatment, at block 104. In some embodiments, the microstates are measured during the treatment session 103. In some embodiments, the microstates are measured up to 2 hours, for example up to 1 hour, up to 30 minutes, or any intermediate, shorter or longer time period, following the delivery of the TMS treatment at block 102. In some embodiments, microstates are measured after the treatment session based on signals, for example EEG signals recorded before and after the delivery of the TMS treatment at block 102, optionally during the treatment session 103.

According to some exemplary embodiments, at least one side effect of the TMS treatment is optionally identified at block 106. In some embodiments, the at least one side effect of the TMS treatment is optionally identified during the TMS treatment 102, during the treatment session 103, or after the treatment session 103. In some embodiments, the at least one TMS treatment side effect comprises at least one of, headache, mild headache, lightheadedness, scalp pain, neck pain, tingling, facial twitching, sleepiness and altered cognition during treatment. In some embodiments, the at least one side effect is optionally identified based on feedback received from the patient.

According to some exemplary embodiments, a short-term effect of the TMS treatment is determined at block 108. In some embodiments, the short term effect is determined based on the microstates measurements performed at block 104, for example based on microstates measurements performed on signals recorded before and after the delivery of the TMS. In some embodiments, the short-term effect is determined based on a change in at least one microstate parameter following the treatment. In some embodiments, the short term-effect is determined by determining a relation between values of the at least one parameter measured after the TMS, and values of the at least one parameter, measured prior to the TMS treatment. Optionally, the values of the at least one microstates parameter measured prior to the TMS treatment are measured prior to the treatment session 103, for example in a baseline visit of the client.

According to some exemplary embodiments, the short-term effect of the TMS treatment is determined by determining a relation between a duration of at least one microstate measured after the TMS treatment, and duration of at least one microstate measured prior to the TMS treatment. In some embodiments, a reduction in duration of at least one microstate indicates a positive short-term effect of the TMS treatment. In some embodiments, a score for a specific TMS protocol is calculated based on the determined short-term effect associated with the TMS protocol.

According to some exemplary embodiments, an indication, for example a human detectable indication is delivered with information regarding the determined intermediate effect. Optionally, the indication comprises instructions to stop the TMS treatment, replace the current TMS protocol, modify the current TMS protocol or modify the TMS treatment, based on the determined short-term effect. Optionally, the indication comprises the score calculated for the TMS protocol.

According to some exemplary embodiments, microstates are measured based on signals recorded after the treatment session, at block 110. In some embodiments, the microstates, for example the at least one parameter of the microstates is measured based on signals, for example EEG signals, recorded after the treatment session 103. In some embodiments, the microstates are measured at bock 110 while the patient is at his home and/or outside the clinic.

According to some exemplary embodiments, a cognitive examination of the patient is performed at block 112. In some embodiments, the cognitive examination is performed using at least one cognitive test given to the patient. In some embodiments, the at least one cognitive test comprises a Mini-Mental State Examination (MMSE, Folstein test), automatized series backwards test, automatized series forward test, Beck Anxiety Inventory test, Beck depression inventory test, Montreal Cognitive Assessment (MoCA) test, Quality of Life in Alzheimer's Disease (QOL-AD) test, recall test, for example an immediate recall test, long delay recall test, stroop raw test, trail making test, and Wechsler Adult Intelligence Scale (WAIS) test, and Computerized tests, for example TABCAT. In some embodiments, the cognitive test is performed at least 24 hours, for example at least 48 hours, at least 72 hours or any intermediate, shorter or longer time period, after the completion of a treatment session in which TMS is delivered at block 102. In some embodiments, the cognitive examination is performed between treatment sessions. Optionally, the cognitive examination is performed after 2, 3, 4, 5 treatment sessions. Additionally or optionally, the cognitive examination is performed prior to an additional treatment session.

According to some exemplary embodiments, an intermediate effect of the TMS delivery is determined at block 114. In some embodiments, determining an intermediate effect of the TMS delivery comprises determining an intermediate effect of a specific TMS protocol during the TMS delivery at block 102. In some embodiments, the intermediate effect is determined prior to an additional treatment session, for example to determine whether to use the same current TMS protocol in the additional treatment session or to replace the current TMS protocol with a new or modified TMS protocol, to stop the TMS treatment or to modify the TMS treatment.

According to some exemplary embodiments, the intermediate effect is determined based on the results of the microstates measurements performed at block 110 and/or based on the results of the cognitive examination performed at block 112. In some embodiments, a score for a TMS protocol, for example a current TMS protocol, is calculated based on the determined intermediate effect. Alternatively, the score is calculated based on the determined intermediate effect and the determined short-term effect of the protocol. In some embodiments, an indication, for example a human detectable indication is delivered with information regarding the determined intermediate effect. Optionally, the indication comprises instructions to stop the TMS treatment, replace the current TMS protocol, modify the current TMS protocol or modify the TMS treatment, based on the determined intermediate effect. Optionally, the indication comprises the score calculated for the TMS protocol.

According to some exemplary embodiments, brain responsiveness to the TMS treatment is optionally estimated at block 116. In some embodiments, the brain responsiveness to the TMS treatment is estimated based on the determined intermediate effect. Alternatively, the brain responsiveness to the treatment is estimated based on the determined intermediate effect and the determined short-term effect. In some embodiments, estimating a brain responsiveness to the treatment comprises estimating whether delivery of an additional TMS will improve a current brain state and/or one or more cognitive abilities, for example cognitive domains or cognitive functionalities, in a subject, and to what extent relative to a current state, In some embodiments, estimating brain responsiveness comprises estimating that an improvement in a brain state and/or cognitive abilities in a subject reached a plateau, and therefore delivering an additional TMS treatment session will not result with a desired additional improvement. Optionally, estimating brain responsiveness comprises estimating a degree of improvement in brain state and/or cognitive abilities following an additional TMS treatment session.

According to some exemplary embodiments, an indication, for example a human detectable indication is delivered with information regarding the estimated brain responsiveness. In some embodiments, the human detectable indication is a graphical indication. Optionally, the indication comprises instructions to stop the TMS treatment, replace the current TMS protocol, modify the current TMS protocol or modify the TMS treatment, based on the estimated brain responsiveness.

According to some exemplary embodiments, the TMS protocol is optionally modified or optionally replaced at block 118. In some embodiments, the TMS protocol used in a previous TMS treatment session is replaced or modified, based on at least one of, the side effect identified at block 106, the short term effect determined at block 108, the intermediate effect determined at block 114 and the brain responsiveness estimated at block 116. In some embodiments, modifying the TMS protocol comprises modifying at least one parameter of the TMS protocol, for example modifying at least one of, coil position on a head of the subject, coil type, coil size, stimulation intensity, stimulation frequency, number of pulses in a train of pulses, number of trains in a treatment session, interval between trains of pulses and overall time of the treatment session.

According to some exemplary embodiments, the TMS treatment is optionally modified at block 120. In some embodiments, modifying the TMS treatment comprises modifying the number of treatment session, for example increasing or decreasing the number of treatment session. Alternatively or additionally, modifying the TMS treatment comprises changing an interval between consecutive treatment sessions, for example shortening or prolonging an interval between consecutive treatment sessions. In some embodiments, the TMS treatment is optionally modified at block 120, based on at least one of, the side effect identified at block 106, the short term effect determined at block 108, the intermediate effect determined at block 114 and the brain responsiveness estimated at block 116. In some embodiments, modifying the TMS treatment comprises combining the TMS treatment with administration of at least one drug.

According to some exemplary embodiments, the TMS treatment is optionally stopped, at block 122. In some embodiments, the TMS treatment is optionally stopped at block 122, based on at least one of, the side effect identified at block 106, the short term effect determined at block 108, the intermediate effect determined at block 114 and the brain responsiveness estimated at block 116.

### Exemplary Detailed Process for Evaluating TMS Treatment

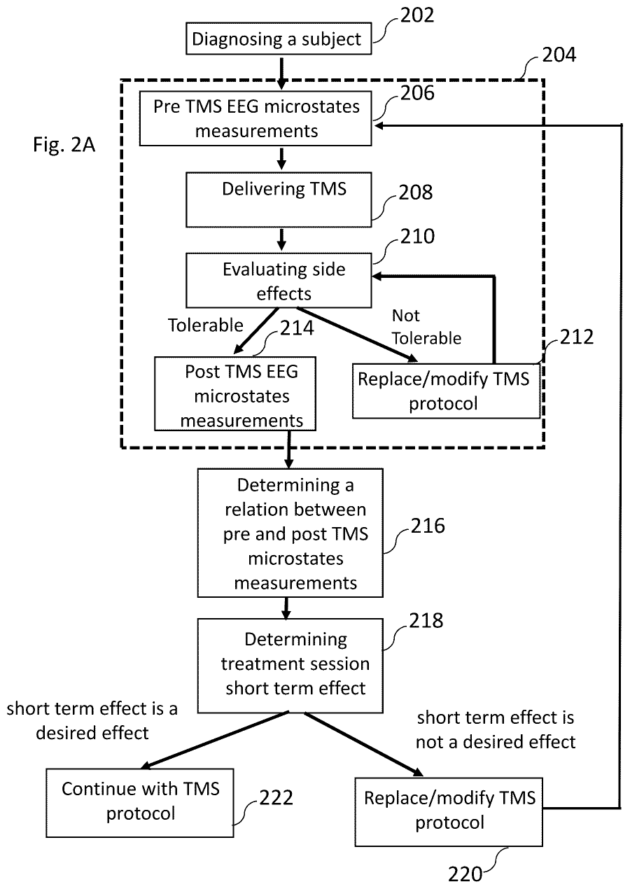

Reference is now made to FIGS. 2A and 2B, depicting a detailed process for evaluating a TMS treatment, according to some exemplary embodiments of the invention.

According to some exemplary embodiments, a subject is diagnosed at block 202. In some embodiments, the subject is diagnosed at block 202 using at least one cognitive exam and/or using at least one imaging technique, for example Magnetic Resonance Imaging (MRI), Computed Tomography (CT), Ultrasound (US), x-ray, and/or nuclear medicine imaging. In some embodiments, the subject is diagnosed at block 202 with a neurological disorder, for example with dementia, Alzheimer's disease (AD), mild cognitive impairment (MCI), depression, or any type of a neurological disorder. Alternatively or additionally, the subject is diagnosed based on microstates measurements, for example based on at least one parameter of microstate. In some embodiments, the at least one parameter comprises duration of at least one microstate.

According to some exemplary embodiments, during diagnosis at block 202, impairment in at least one functional cognitive domain is identified. In some embodiments, during or following the diagnosis, a treatment, for example a tMS treatment or a pharmaceutical treatment is selected to treat the diagnosed neurological disorder and/or to improve the impairment in the at least one functional cognitive domain. Optionally, a plan for a treatment procedure is generated following or during the diagnosis. In some embodiments, the plan of the treatment procedure comprises a number of treatment sessions, interval between treatment sessions, timing for measuring microstates and/or for performing at least one cognitive exam, for example between treatment sessions. In some embodiments, the treatment procedure plan comprises selecting an initial, for example a first treatment protocol, for example a TMS protocol.

According to some exemplary embodiments, pre-TMS EEG microstates are measured at block 206, optionally prior to TMS delivery. In some embodiments, at least one parameter of the microstates is measured at block 206. In some embodiments, the at least one microstates parameter comprises duration of at least one microstate. In some embodiments, the pre-TMS EEG microstates are measured using EEG signals recorded prior to TMS delivery. In some embodiments, the EEG signals and/or the microstates are measured as during a TMS treatment session 204.

According to some exemplary embodiments, TMS is delivered at block 208. In some embodiments, the TMS delivered according to a least one TMS protocol, for example a TMS protocol selected based on the diagnosis at block 202. In some embodiments, the TMS comprises repetitive TMS (rTMS). Optionally, the TMS comprises deep TMS which allows penetration of the stimulation at least 2 cm, for example at least 4 cm or any intermediate, smaller or larger depth, underneath the skull surface.

According to some exemplary embodiments, side effects or severity thereof, of the delivered TMS are evaluated at block 210. In some embodiments, the side effects are evaluated based on input received from the patient. Alternatively or additionally, the side effects are evaluated based on measurements of at least one physiological or clinical parameter, for example heart rate or blood pressure. In some embodiments, the side effects comprise headache, pain, and/or twitching.

According to some exemplary embodiments, if the evaluated side effects are not tolerable, the TMS protocol is replaced at block 212. Alternatively, the TMS protocol used at block or at least one parameter of the TMS protocol is modified. In some embodiments, the at least one parameter comprises location of the stimulation, intensity of the stimulation, number of pulses per train, number of trains, duration of each pulse or duration of each train, and/or overall duration of a treatment session.

According to some exemplary embodiments, if the evaluated side effects are tolerable, post-TMS EEG microstates are measured, at block 214. In some embodiments, the post-TMS microstates are measured based on EEG signals recorded following the delivery of TMS during the treatment session 202, for example up to 2 hours, up to 1 hour up to 30 minutes, up to 20 minutes from completing the delivery of TMS to the patient.

According to some exemplary embodiments, a relation between pre and post TMS microstates measurements is determined at block 216, for example by a control circuitry of a microstates measurement device. In some embodiments, the relation is determined between indications of pre-TMS microstates measurements measured at block 206 and indications of post-TMS measurements, measured at block 214. In some embodiments, the indications are stored in a memory of the device.

According to some exemplary embodiments, determining a relation between pre and post TMS microstates measurements comprises calculating a change in at least one microstate parameter, for example microstate duration, frequency, Transitions, Coverage and the delta of the average for each parameter, following the delivery of the TMS at block 208. In some embodiments, calculating a change comprises calculating a change or measures of central tendency, for example average, mean or median, of the at least one microstates parameter, following TMS delivery.

According to some exemplary embodiments, a short-term effect of the treatment session 204 is determined at block 218. In some embodiments, the short-term effect is determined based on the determined relation between pre and post TMS microstates measurements. In some embodiments, the determined short-term effect is an effect of a specific TMS protocol used in the treatment session. In some embodiments, the short term effect is determined based on the change in the at least one microstates parameter calculated at block 216.

According to some exemplary embodiments, if the short term effect is a desired effect, for example a target effect, then the TMS protocol remains unchanged and will be used in additional treatment sessions, at block 222. Alternatively, if the short-term effect is not a desired effect, the TMS protocol is modified or replaced with a different TMS protocol. In some embodiments, a target short-term effect comprises a desired improvement of the patient condition, for example a desired improvement of a brain state and/or a desired improvement of at least one cognitive function of the patient following the treatment session 204, for example up to 48 hours, up to 24 hours, up to 12 hours or any intermediate, smaller or larger time period following the treatment session 204. In some embodiments, a change in the at least one microstates parameter indicates the desired improvement of the patient condition. In some embodiments, a reduction in at least 10%, for example at least 15%, at least 25%, at least 40%, at least 50% or any intermediate, smaller or larger percentage value of reduction in a duration of at least one microstate, is a desired change indicating a desired improvement of the patient condition following the treatment session 204.

According to some exemplary embodiments, if the reduction in the duration of the at least one microstate is smaller than 10%, smaller than 5%, smaller than 1% or any intermediate, smaller or larger percentage value, or if there is no reduction, the TMS protocol is replaced or modified at block 220. In some embodiments, the at least one microstate comprises at least one of microstates A, B, C, and D.

Reference is now made to FIG. 2B, depicting evaluating a TMS protocol based on a prolonged effect, for example an intermediate effect of at least one treatment session using the TMS protocol, according to some exemplary embodiments of the invention.

According to some exemplary embodiments, EEG microstates are measured at block 224. In some embodiments, measurements of EEG microstates comprise measurements of at least one microstates parameter or measures of central tendency of the at least one parameter. In some embodiments, the EEG microstates are measured at least 48 hours, for example at least 72 hours, at least 5 days, at least a week, or any intermediate, smaller or larger time period, following the treatment session 204. In some embodiments, the EEG microstates are measured at block 224 prior to an additional treatment session, or between two consecutive treatment session of a treatment procedure. In some embodiments, the EEG microstates are measured at block 224 using EEG signals recorded at least 48 hours, for example at least 72 hours, at least 5 days, at least a week, or any intermediate, smaller or larger time period, following the treatment session 204. In some embodiments, the EEG microstates are measured at block 224 using EEG signals recorded prior to an additional treatment session, or between two consecutive treatment session of a treatment procedure.

According to some exemplary embodiments, at least one cognitive exam is optionally performed at block 226. In some embodiments, the at least one cognitive exam is performed, for example as described at block 112 shown in FIG. 1B. In some embodiments, the at least one cognitive exam is optionally performed at least 48 hours, for example at least 72 hours, at least 5 days, at least a week, or any intermediate, smaller or larger time period, following the treatment session 204. In some embodiments, the at least one cognitive exam is optionally performed at block 226 prior to an additional treatment session, or between two consecutive treatment session of a treatment procedure.

According to some exemplary embodiments, a relation between the microstates measurements or indications thereof, performed at the end of the treatment session, at block 214, and the microstates measurements or indications thereof, performed at block 224 is determined at block 228. In some embodiments, the relation is determined by a control circuitry of a microstates measurements device.

According to some exemplary embodiments, determining a relation between microstates measurements performed at an end of a treatment session, and microstates measurements performed prior to an additional treatment session or between treatment sessions comprises calculating a change in at least one microstate parameter, for example duration of at least one microstate, frequency, transitions, coverage and the delta of the average for each parameter, at least 48 hours following the delivery of the TMS at block 208. In some embodiments, calculating a change comprises calculating a change or measures of central tendency, for example average, mean or median, of the at least one microstates parameter, following TMS delivery.

According to some exemplary embodiments, a prolonged effect, for example an intermediate effect of the treatment session 204 is determined at block 230. In some embodiments, the prolonged effect is determined based on the relation determined at block 228. Alternatively, the prolonged effect is determined based on the relation determined at block 228 and the results of the cognitive exams performed at block 226. In some embodiments, the determined prolonged effect is a prolonged effect of a specific TMS protocol used in the treatment session. In some embodiments, the prolonged effect is determined based on the change in the at least one microstates parameter calculated at block 216.

According to some exemplary embodiments, if the prolonged effect is a desired effect, for example a target effect, then the TMS protocol remains unchanged and will be used in additional treatment sessions, at block 232. Alternatively, if the prolonged effect is not a desired effect, the TMS protocol is modified or replaced with a different TMS protocol at block 234. In some embodiments, a target prolonged effect comprises a desired improvement of the patient condition, for example a desired improvement of a brain state and/or a desired improvement of at least one cognitive function of the patient following the treatment session 204, for example more than 48 hours, more than 72 hours, more than 5 days, more than a week or any intermediate, smaller or larger time period following the treatment session 204. In some embodiments, a change in the at least one microstates parameter indicates the desired improvement of the patient condition. In some embodiments, a reduction in at least 10%, for example at least 15%, at least 25%, at least 40%, at least 50% or any intermediate, smaller or larger percentage value of reduction in a duration of at least one microstate, is a desired change indicating a desired improvement of the patient condition following the treatment session 204.

According to some exemplary embodiments, if the reduction in the duration of the at least one microstate is smaller than 10%, smaller than 5%, smaller than 1% or any intermediate, smaller or larger percentage value, or if there is no reduction, the TMS protocol is replaced or modified at block 234. In some embodiments, the at least one microstate comprises at least one of microstates A, B, C, and D.

Alternatively or additionally, if there is no improvement or the improvement is not a desired improvement, in the results of the cognitive exams performed at block 226 in comparison to the cognitive exams performed at block 202 or prior to the treatment session 204, the TMS protocol is replaced or modified.

### Exemplary Process for Selecting a TMS Protocol

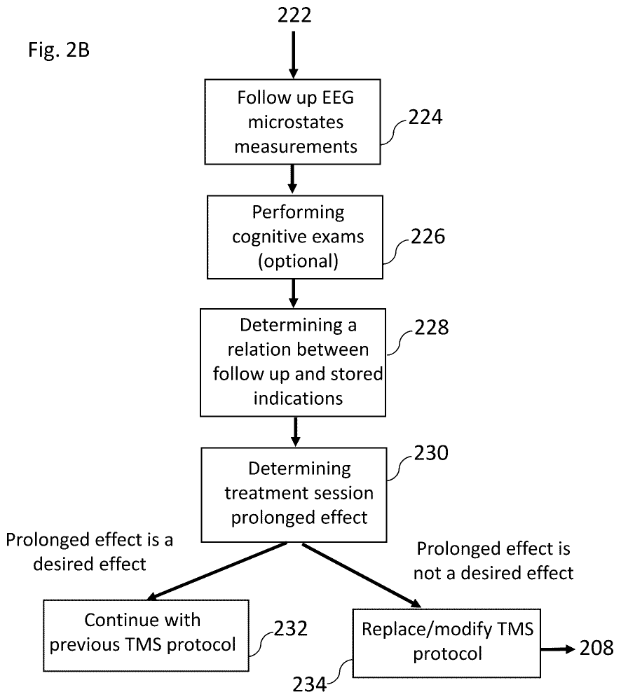

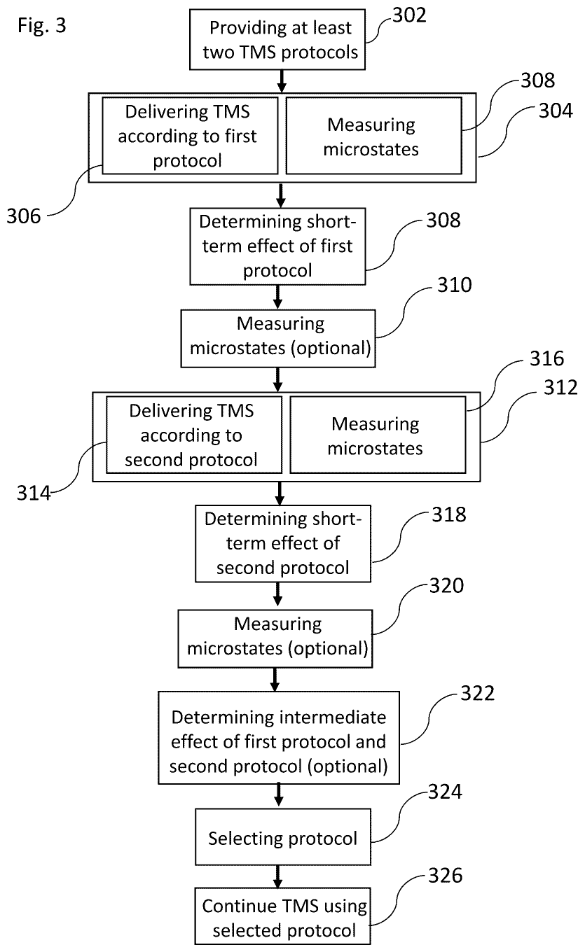

Reference is now made to FIG. 3, depicting a process for selecting a TMS protocol, according to some exemplary embodiments of the invention.

According to some exemplary embodiments, at least two TMS protocols are provided, at block 302.

According to some exemplary embodiments, at least one first TMS protocol of the at least two TMS protocol, is used in a first treatment session, at block 304. In some embodiments, TMS is delivered to a patient at block 306 according to the at least one first TMS protocol, at block 306. In some embodiments, microstates are measured before and after the TMS delivery using the at least one first TMS protocol, at block 308. In some embodiments, at least one microstates parameter is measured. In some embodiments, the microstates are measured at block 308 using EEG signals recorded before and after the delivery of TMS 308. Optionally, the EEG signals and/or the microstates are measured while the patient is still at the clinic. Optionally, EEG signals are recorded up to 2 hours, for example up to 1 hour, up to 30 minutes, up to 20 minutes or any intermediate, smaller or larger time period, after the delivery of the TMS at block 306.

According to some exemplary embodiments, a short term effect of the TMS delivered at block 306 using the at least one first TMS protocol is determined at block 308. In some embodiments, determining a short term effect of the treatment comprises calculating a change in at least one parameter of the microstates following the TMS delivery, for example calculating a change in duration of at least one microstate, following the delivery of the TMS at block 306, compared to a value or an indication thereof, measured prior to the TMS delivery at block 306. In some embodiments, a short-term effect of the TMS treatment relates to an immediate effect of the treatment on measurements of at least one parameter of the microstates, indicating an effect of the treatment on a brain state of the patient.

According to some exemplary embodiments, the short-term effect determined at block 308 is associated with the at least one first TMS protocol. Optionally, the determined short-term effect, for example the calculated change in the at least one microstates parameter is used to score and/or rank the at least one first TMS protocol.

According to some exemplary embodiments, microstates, for example at least one parameter of the microstates, are optionally measured at block 310. In some embodiments, the microstates are measured at block 310 between two treatment sessions of the TMS treatment, for example to determine an intermediate effect of the TMS delivered at block 306 and/or the intermediate effect of the TMS protocol used for the TMS delivery at block 306. In some embodiments, the microstates are optionally measured at block 310 using EEG signals recorded at least 24 hours, for example at least 48 hours, at least 72 hours or any intermediate, smaller or larger time period from completing the treatment session 304. Optionally, the EEG signals are recorded while the patient is outside the clinic, for example at his home, and are transmitted to a remote device for measuring the microstates. Alternatively, the microstates are measured locally using the patient device.

According to some exemplary embodiments, the patient undergoes at least one additional treatment session at block 312. In some embodiments, during the treatment session, TMS is delivered using at least one second TMS protocol of the at least two TMS protocols provided at block 302. In some embodiments, microstates, for example at least one microstates parameter, are measured before and after the TMS delivery, at block 316, for example as described with respect to the microstates measurements at block 308. In some embodiments, the microstates are measured at block 316 using EEG signals recorded prior to and According to some exemplary embodiments, a short-term effect of the TMS delivery at block 314, using the at least one second TMS protocol, is determined at block 318/In some embodiments, the short-term effect is determined at block 318, for example as described at block 308.

According to some exemplary embodiments, the short-term effect determined at block 318 is associated with the at least one second TMS protocol. Optionally, the determined short-term effect, for example the calculated change in the at least one microstates parameter is used to score and/or rank the at least one second TMS protocol.

According to some exemplary embodiments, microstates, for example at least one parameter of the microstates, are optionally measured at block 320. In some embodiments, the microstates are measured at least 24 hours after completing the second treatment session 312. In some embodiments, the microstates are measured using EEG signals recorded at least 24 hours after completing the second treatment session 312. In some embodiments, the microstates are measured at block 320, between two TMS treatment sessions, for example as described at block 310.

According to some exemplary embodiments, an intermediate effect of the at least one first TMS protocol and the at least one second TMS protocol, is optionally determined at block 322. In some embodiments, an intermediate effect is an effect of a TMS treatment provided using at least one specific TMS protocol after a time period of at least 24 hours after completing the TMS treatment session.

According to some exemplary embodiments, the intermediate effect of each TMS protocol is determined by calculating a change in a value of at least one microstates parameter after at least 24 hours from completing the treatment session, compared to a value of the at least one microstates parameter measured prior to the TMS delivery using the specific treatment protocol. In some embodiments, an intermediate effect is an effect of a TMS treatment protocol measured during a treatment process, for example between treatment sessions. In some embodiments, the intermediate effect of each TMS protocol is used to score and/or rank the TMS protocol compared to other TMS protocols.

According to some exemplary embodiments, an overall score for each TMS protocol is calculated based on a combination between the short-term effect and the intermediate term effect of each TMS protocol. Optionally, side effects or severity of side effects associated with each TMS protocol are combined with the short-term effect and the intermediate effect of each TMS protocol, when generating an overall score of a TMS treatment protocol.

According to some exemplary embodiments, a TMS protocol is selected at block 324. In some embodiments, a TMS protocol is selected for a following at least one additional treatment session of a TMS treatment procedure, at block 324. In some embodiments, the TMS protocol is selected based on the determined short-term effect of each of the at least two TMS protocols provided at block 302. Alternatively or additionally, the TMS protocol is selected based on the determined intermediate effect of each of the at least two TMS protocols provided at block 302. Alternatively, the TMS protocol is selected based on the overall score for each of the at least two TMS protocols generated by adjusted calculation of the short-term effect, the intermediate effect and optionally the severity of side effects associated with each protocol.

According to some exemplary embodiments, the TMS procedure continues with the selected TMS protocol, at block 326. In some embodiments, further microstates measurements and/or cognitive exams are used to determine whether to continue with the selected TMS protocol or to replace the selected TMS protocol with a different TMS protocol, or to modify at least one parameter of the selected protocol. Optionally, the further microstates measurements and/or cognitive exams are used to update a score of each TMS protocol.

### Exemplary Device for Microstates Measurements

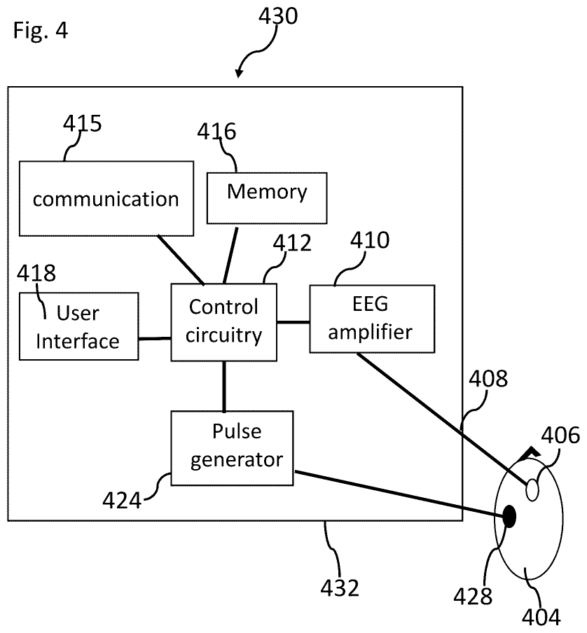

According to some exemplary embodiments, a device is used for measurements of microstates, for example for measurements of at least one parameter for example a feature of microstates, during a TMS treatment process. Reference is now made to FIG. 4, depicting a device for microstates measurements, according to some exemplary embodiments of the invention.

According to some exemplary embodiments, a device for microstates measurements, for example device 430 comprises a control circuitry 412, which is connectable through an EEG amplifier 410 to at least one electrode 406 positioned on a head 404 of a subject. In some embodiments, the control circuitry 412 is configured to receive EEG signals via the at least one electrode 406 and the amplifier 410. In some embodiments, the control circuitry 412 records the EEG signals and stores the EEG signals or indications thereof in memory 416.

According to some exemplary embodiments, the device 430 EEG signals during a TMS treatment session, for example before and after the delivery of TMS to a patient. In some embodiments, the control circuitry 412 analyzes the stored pre-TMS EEG signals and the post-TMS EEG signals to extract at least one parameter, for example feature, of microstates from the stored EEG signals. In some embodiments, the control circuitry 412 calculates a change in values for the at least one microstates parameter between the post-TMS EEG signals and the pre-TMS EEG signals. In some embodiments, the control circuitry 412 determines a short-term effect of at least one TMS protocol used for the TMS treatment based on the calculated change in the values of the at least one microstates parameter. In some embodiments, during the analysis, the control circuitry 412 generates a score for the determined short-term effect. In some embodiments, the control circuitry 412 analyzes the stored EEG signals for extracting the at least one microstates parameter, for calculating the change in the at least one microstate parameter value, to determine a short-term effect of a TMS protocol and/or to generate a score for the determined short-term effect, using at least one algorithm, formula, and/or lookup table stored in the memory 416.

According to some exemplary embodiments, the device 430 comprises a communication circuitry 415 for receiving and/or transmitting signals, for example wireless signals between the device 430 and at least one remote device. In some embodiments, the remote device comprises at least one of, a remote computer, a remote database, a remote cloud storage, a mobile device and a cellular device. In some embodiments, a remote device is a device located at a distance of at least one meter from the device 430, and optionally outside a room in which the device 430 is located.

According to some exemplary embodiments, the device 430 transmits the recorded EEG signals stored in the memory 416 to the remote device for analysis, using the communication circuitry 415. In some embodiments, the remote device analyzes the EEG signals using at least one algorithm, formula, and/or lookup table stored in a memory of the remote device. In some embodiments, the remote device stores the analysis results in a memory of the remote device. Alternatively or additionally, the device 430 receives from the remote device the results of the analysis or indications thereof, for example the short-term effect, a score for the determined short-term effect.

According to some exemplary embodiments, the device 430 analyzes the received EEG signals to determine an intermediate effect of a TMS delivery using a specific TMS protocol, using EEG signals recorded prior to the TMS delivery or immediately after the TMS delivery, for example up to 12 hours following the TMS delivery, and EEG signals recorded post treatment session, at least 24 hours following the TMS delivery and prior to an additional treatment session.

According to some exemplary embodiments, the control circuitry 412 analyzes the stored EEG signals to extract the at least one parameter, for example the feature, of microstates from the stored EEG signals. In some embodiments, the control circuitry 412 calculates a change in values for the at least one microstates following the treatment session, for example at least 24 hours following the treatment session. In some embodiments, the control circuitry 412 determines an intermediate effect of at least one TMS protocol used for the TMS treatment based on the calculated change in the values of the at least one microstates parameter. In some embodiments, during the analysis, the control circuitry 412 generates a score for the determined intermediate effect. In some embodiments, the control circuitry 412 analyzes the stored EEG signals for extracting the at least one microstates parameter, for calculating the change in the at least one microstate parameter value, to determine the intermediate effect of a TMS protocol and/or to generate a score for the determined intermediate effect, using at least one algorithm, formula, and/or lookup table stored in the memory 416. Alternatively, as previously described with respect to the short-term effect, a remote device performs the analysis of the EEG signals in order to determine the intermediate effect, and delivers the analysis results or indications thereof to the device 430 via the communication circuitry.

According to some exemplary embodiments, the device 430 comprises a user interface 418, configured to generate and deliver an indication, for example a human detectable indication. In some embodiments, the indication comprises an audio and/or a visual indication. Optionally, the user interface 418 comprises a display, and the user interface 418 delivers the indication using the display. In some embodiments, the control circuitry 412 signals the user interface 418 to generate and deliver a human detectable indication with the results of the analysis of the EEG signals, for example with indication or information regarding at least one of the determined short-term effect, the intermediate effect and/or a score associated with at least one TMS protocol which is calculated based on the short-term effect and the intermediate effect.

According to some exemplary embodiments, the user interface 418 comprises at least one input module, for example a touch screen, or a keyboard, for receiving information with regard to results of at least one cognitive exam, and/or to receive information regarding at least one side effect of at least one TMS protocol. Optionally, the control circuitry 412 generates a weighted score for each TMS protocol based on the determined short-term and/or the determined intermediate effect of the TMS protocol, the results of the cognitive exam performed following the TMS treatment and/or an input from the patient with regard to side effects of the at least one TMS protocol.

According to some exemplary embodiments, the control circuitry 412 signals the user interface 418 to generate and deliver an indication with suggestions and/or recommendations which TMS protocol to select for following TMS treatment sessions, based on at least one of, the generated weighted score, the determined short-term effect or score thereof, the intermediate effect or score thereof, the results of cognitive exams entered into the device 430 and the side effects associated with each TMS protocol.

According to some separate exemplary embodiments, the device is not only used for receiving EEG signals, but it is also used for delivery of the TMS to the patient. In some embodiments, the device comprises a pulse generator 424 in functional communication with the control circuitry 412. In some embodiments, the memory 416 stores at least one TMS protocol or parameters thereof. In some embodiments, the pulse generator is connectable to at least one magnetic coil 428 configured to be positioned close to, or in contact with a head of the patient 404. In some embodiments, the control circuitry 412 signals the pulse generator 424 to generate a magnetic stimulation according to the at least one TMS protocol and/or parameters thereof stored in the memory 416.

According to some exemplary embodiments, the device synchronizes the delivery of TMS with the recording of the EEG signals before or after the delivery of TMS to the patient 404.

According to some exemplary embodiments, a control circuitry of a system for delivery of a TMS treatment and/or of a system for determining an efficacy of a TMS treatment, for example the control circuitry 412, is configured and operable to perform one or more of method steps described in FIGS. 1A, 1B, 2A, 2B, 3, 5, 6A, 6B, 7, 8, and 9.

### Exemplary Method for Personalizing a TMS Protocol

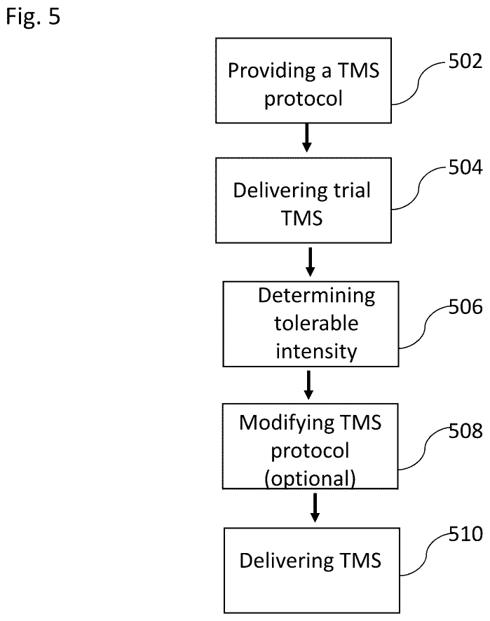

Reference is now made to FIG. 5 depicting a process for personalizing a TMS protocol, according to some exemplary embodiments of the invention. In some embodiments, the TMS is a repetitive TMS, and is optionally a deep TMS configured to penetrate to a depth of up to 4 cm below a surface of the skull.

According to some exemplary embodiments, a TMS protocol, is provided at block 502. In some embodiments, the TMS protocol comprises at least one parameter of the magnetic stimulation and a location for delivering of the stimulation. In some embodiments, the magnetic stimulation has a frequency of about 10 Hz, with an intensity, for example of about 100% of a predetermined motor cortex intensity threshold, duration of each train of pulses is about 2 seconds, number of pulses per train is about 20 pulses, wait time between each train of pulses is about 20 seconds, and an overall number of trains of pulses is about 50. Additionally, in some embodiments, a stimulation location is over a dorsolateral prefrontal cortex. Optionally, the stimulation location is about 4 cm anterior to a location on a scalp which is the closest location to a primary motor cortex in a subject, that optionally generates the maximal muscle twitch of a subject fingers.

According to some exemplary embodiments, a trial TMS stimulation is delivered at block 504. In some embodiments, the trial TMS stimulation is delivered using the parameters of the TMS protocol described at block 502.

According to some exemplary embodiments, a tolerable intensity of the TMS protocol per subject is determined, at block 506. In some embodiments, the tolerable intensity is an intensity level that does not generate pain sensation, or that generates a tolerable level of pain sensation in the subject. In some embodiments, the tolerable intensity is determined by receiving input from the patient following the delivery of at least one trial TMS at block 504. In some embodiments, a tolerable intensity is within a range of up to about 10%, for example up to about 5%, up to about 1% lower than the predetermined motor cortex intensity.

According to some exemplary embodiments, the TMS protocol is optionally modified at block 508. In some embodiments, the TMS protocol is optionally modified by adjusting the intensity, for example the power, of the magnetic stimulation to the intensity determined at block 506.

According to some exemplary embodiments, a TMS treatment is delivered to the subject, at block 510 optionally using the modified TMS protocol.

Exemplary microstates extraction and features analysis

According to some exemplary embodiments, microstates are measured, for example extracted from EEG signals as previously described in WO2019/064136 filed on 21 Sep. 2018, incorporated herein as a reference in its entirety.

Reference is now made to FIG. 6A depicting a process for extraction of microstates from one or more EEG signals that was used in an experiment and in some embodiments of the invention. In some embodiments, the extraction process is performed using the device 430 shown in FIG. 4.

In some embodiments and in the experiment, EEG signals in a frequency range of 1-40 Hertz were filtered at block 604. In some embodiments of the invention, EEG signals in a frequency range of 1-40 Hertz, for example 1-10, 2-20, 2-25 or any intermediate, larger range or smaller, were filtered. Optionally, in some embodiments of the invention, EEG signals in frequencies larger than 40 Hertz and/or smaller than 1 Hertz are filtered.

In some embodiments and in the experiment, quiet segments in the filtered EEG signals were extracted at block 606. In the experiment and in some embodiments of the invention, quiet segments are segments where the EEG signals are resting state EEG signals. In the experiment and in some embodiments of the invention, quiet segments are segments in the EEG recorded when the eyes of a subject are closed. In the experiment, all EEG signals were resting state EEG. The relevant segments used for microstate extraction were those segments when eyes were closed. In the experiment, an algorithm extracted those segments when alpha rhythm appears (i.e eyes closed), for example for automatically extraction of microstates. In some embodiments of the invention, as used in the experiment, quiet segments are identified and optionally extracted based on the detection of an alpha rhythm. Optionally, in some embodiments of the invention, detection of an alpha rhythm and/or extraction of the quiet segments are performed by one or more algorithms, for example one or more algorithms stored in a memory.

In some embodiments of the invention, the presence and/or levels of the alpha rhythm is monitored, optionally continuously monitored. In some embodiments, the presence and/or levels of the alpha rhythm are monitored during a specific time period, for example during a time period between 1 minute and 60 minute, or any other larger or smaller time period. In some embodiments, when the levels of the alpha rhythm are higher than a predetermined level the EEG signals are recorded and optionally are classified as resting state EEG signals. In some embodiments of the invention, a system monitors the eyes closure rate, or any other clinical or physiological parameter indicative of a resting state.

In some embodiments and in the experiment, Global Field Power (GFP) was calculated at block 608.

In some embodiments and in the experiment, microstates were extracted from the filtered EEG signals at block 610. In the experiment and in some embodiments of the invention, the microstates comprise microstate A, microstate B, microstate C and/or microstate D. In some embodiments of the invention, the microstates comprise one or more of microstate A, microstate B, microstate C and/or microstate D or any number of defined microstates, for example the number of microstates or the definition of each microstate is personalized to a specific subject and/or to a specific clinical condition. In some embodiments, microstates are extracted as described in FIG. 6B.

In some embodiments and in the experiment, microstates parameters, also termed herein as “features” were extracted from the microstate vector at block 612. In the experiment and in some embodiments of the invention the microstates parameters are extracted, as described in the “Exemplary microstates extraction” section of this application. In the experiment and in some embodiments of the invention, the features comprise one or more of microstate duration, microstate frequency, microstate coverage and microstate transitions or any mathematical derivation of the features, for example a mean of the features.

In some embodiments of the invention, one or more microstates, for example microstate A and/or microstate B and/or microstate C and/or microstate D are extracted from an EEG signal. In some embodiments, values of one or more microstates parameters are calculated, for example microstate duration and/or microstate frequency and/or microstate coverage and/or number of transitions of one or more microstates or any mathematical derivation of the values, for example a mean or an average.

Reference is now made to FIG. 6B, depicting a process for extraction of microstates parameters from EEG signals, according to some exemplary embodiments of the invention.

According to some exemplary embodiments, EEG parameters are measured at block 603, by at least two electrodes placed in contact with the head of an individual. In some embodiments, at least one electrode serves as a reference electrode to the rest of the electrodes. In some embodiments, EEG parameters are measured during a resting state of the individual. In some embodiments a resting state is achieved when the individual is not actively engaged in sensory and/or cognitive processing, for example when the eyes of the individual are closed. In some embodiments, EEG measures brain waves with frequencies of 1-4 Hertz (delta waves), 4-7 Hertz (theta waves) and/or 8-12 Hertz (alpha waves), 12-28 Hertz (beta waves), and/or >30 Hertz (gamma waves). In some embodiments, EEG parameters are measured as described in Khanna et, al. 2015.

According to some exemplary embodiments, EEG topographic states are extracted from the EEG measurements at block 605. In some embodiments, the EEG signal is analyzed to generate a global field power (GFP) curve. In some embodiments, the GFP curve represents the strength of the electric field over the brain at each instant. In some embodiments, the GFP curve is used to measure the brain response to an event, or to characterize the rapid changes in brain activity. In some embodiments, a local maximum of GFP curve represents instants of strongest field strength and highest topographic signal to noise ratio. In some embodiments, the topographies of the electric field at local maxima of the GFP curve are considered discrete states of the EEG. Optionally, these discrete states are termed EEG microstates.

According to some exemplary embodiments, a clustering analysis of the extracted microstates is performed at block 607. In some embodiments, a clustering algorithm groups the extracted microstates into sets of clusters. In some embodiments, the clustering is based on topographic similarity between some of the extracted microstates. In some embodiments, EEG microstates, for example resting-state EEG microstates are clustered into a set of 2, 3, 4, 5, 6, for example 4 microstate maps.

According to some exemplary embodiments, the frequency of occurrence of each microstate map is determined at block 609. In some embodiments, a single microstate map remains stable for 50-150 milliseconds, for example 80-120 milliseconds before transitioning to a different microstate map. In some embodiments, the occurrence time of each microstate map before its transition into a new microstate map is calculated.

According to some exemplary embodiments, microstate parameters values are determined at 611, based on the clustering analysis of the topographic states and/or based on the frequency of occurrence of each microstate map that was determined at 609. In some embodiments, microstate parameters comprise at least two microstate maps, for example 2, 3, 4 microstate maps, and the occurrence time of each microstate map.

In some embodiments, microstates are extracted from EEG measurements by comparing the topography at each successive GFP peak to the previous GFP peak and consider it as the start of a new microstate if the centroid locations of the positive or negative potentials change by more than a predetermined level (Lehman et al., 1987).

Alternatively, microstates are extracted from EEG measurements by independent component analysis to define microstate classes (Musso et al., 2010; Yuan et al., 2012).

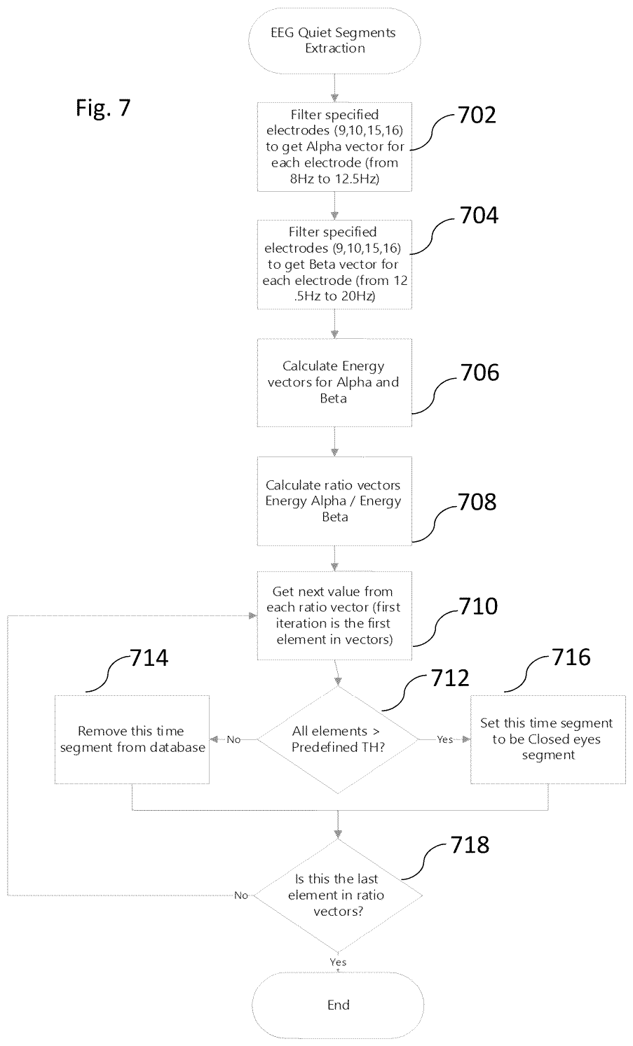

Reference is now made to FIG. 7, depicting a process that was used in the experiment and that can also be used in some embodiments of the invention for extraction of EEG quiet segments.

In some embodiments and in the experiment, EEG signals were filtered from a desired number of electrodes to get an Alpha vector for each electrode at block 702. Optionally, in some embodiments of the invention, EEG signals from 8, 9, 10, 15 16 or any desired number of electrodes are filtered. In some embodiments and in the experiment, an Alpha vector was received by filtering EEG signals in a range of 8 Hertz to 12.5 Hertz. Optionally, in some embodiments of the invention an alpha vector is received in a different range of frequencies, for example a range of frequencies that is personalized for a specific subject or to a specific clinical condition.

In some embodiments and in the experiment, EEG signals were filtered from a desired number of electrodes to get a Beta vector for each electrode at block 704. Optionally, in some embodiments of the invention, EEG signals from 8, 9, 10, 15 16 or any desired number of electrodes are filtered. In some embodiments, a beta vector was received by filtering EEG signals in a range of 12.5 Hertz to 20 Hertz. Optionally, in some embodiments of the invention a beta vector is received in a different range of frequencies, for example a range of frequencies that is personalized for a specific subject or to a specific clinical condition.

In some embodiments and in the experiment, energy vectors for Alpha and/or Beta vectors were calculated at block 706. A ratio between Alpha energy vectors and Beta energy vectors was calculated at block 708.

In some embodiments and in the experiment, the next value from each ratio vector is calculated (first iteration is the first element in vectors) at block 710. If all elements are larger than a predefined threshold as checked at block 712, the specific analyzed time segment was set to be a closed eyes segment at block 716.

In some embodiments and in the experiment, if all elements were not larger than a predefined threshold as checked at block 712, then the specific analyzed time segment was removed at block 714. Optionally, in some embodiments of the invention, a specific analyzed time segment is removed from a database of signals, for example a database of signals that is used for further analysis of the EEG signals.

In some embodiments and in the experiment, if this was not the last element in the ratio vectors as checked at block 718, then the next value from each ratio vector was calculated at block 710.

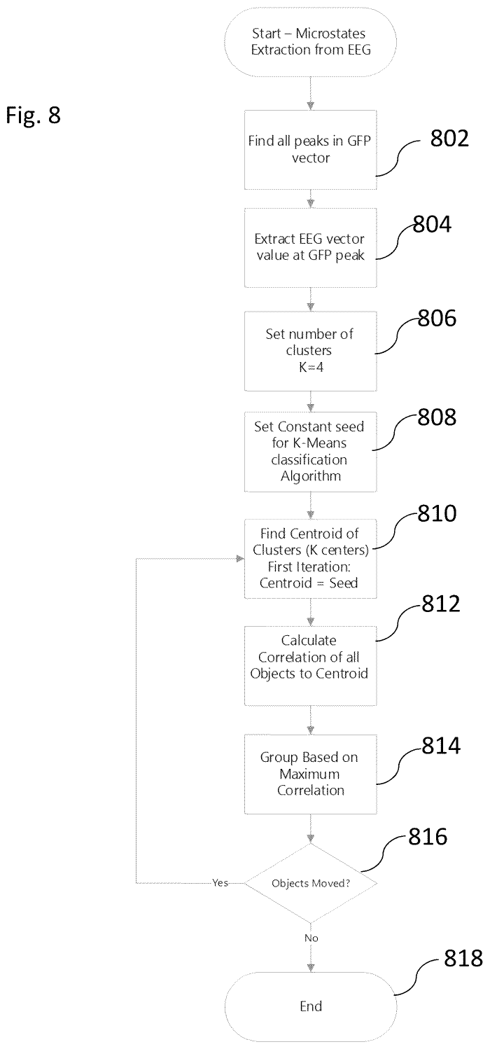

Reference is now made to FIG. 8 depicting a process for microstates extraction as used in the experiment and that can also be used in some embodiments of the invention. In some embodiments and in the experiment, the microstates were extracted from quiet segments of the EEG signal, for example from closed eyes segments of the EEG signal. Optionally, in some embodiments of the invention, the microstates are extracted from closed time segments stored in a database of time segments.

In some embodiments and in the experiment, peaks in the GFP vector were detected at block 802. Optionally, in some embodiments of the invention, only selected peaks in the GFP vector, for example peaks that are larger than a predefined threshold are detected. EEG vector value at each GFP peak was extracted at block. Optionally, in some embodiments of the invention, EEG vector values at selected GFP peaks are extracted.

In some embodiments and in the experiment, the number of clusters was set to k=4 at block 806. In addition, constant seed for k-means classification was set at block 808.

In some embodiments and in the experiment, a centroid of clusters (k centers) was detected at block 810. Optionally, first iteration is centroid=seed.

In some embodiments and in the experiment, a correlation of all objects to the centroid was calculated at block 812. Optionally, in some embodiments of the invention, a correlation of selected objects to the centroid is calculated.

In some embodiments and in the experiment, one or more objects were grouped based on maximum correlation at block 814.

In some embodiments and in the experiment, a check was performed to determine if objects moved at block 816.

In some embodiments and in the experiment, if objects did not move then the process was ended at block 818.

In some embodiments and in the experiment, if objects moved, then a centroid of clusters (k centers) was found as previously described at block 810.

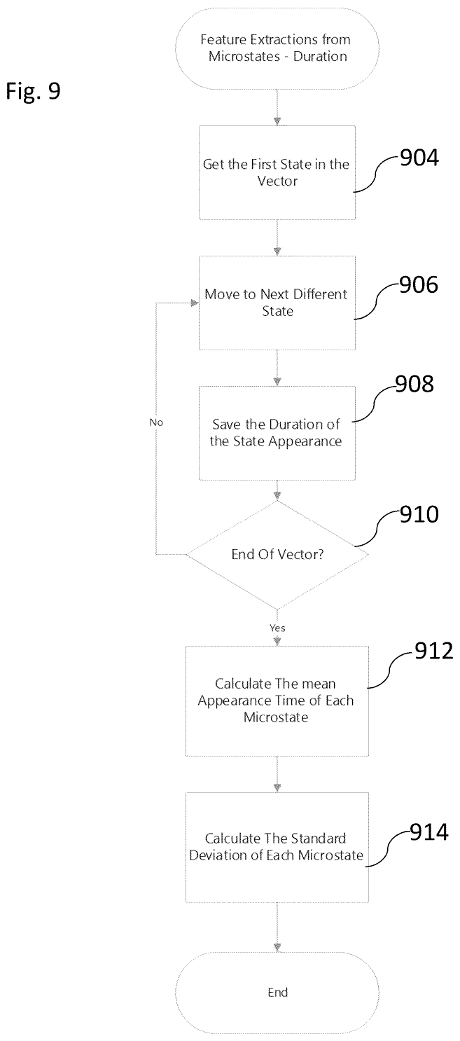

Reference is now made to FIG. 9, depicting a process for extraction of microstates duration as used in the experiment and that can also be used in some embodiments of the invention.

In some embodiments and in the experiment, the first state in the vector was detected at block 904. Additionally, the next different state was detected at block 906. In some embodiments and in the experiment, the duration of the state appearance was saved at block 908. A check was performed to determine if this is the end of the vector at block 910.

In some embodiments and in the experiment, if this was not the end of the vector, then the process moved to the next different state at block 906. In some embodiments and in the experiment, if this was end of the vector, then the mean appearance time of each microstate was calculated at block 912. Additionally or optionally, the standard deviation of each microstate is calculated at block 914.

### Experiment

In an experiment, a TMS treatment was delivered to a group of 12 patients diagnosed with Mild Cognitive Impairment (MCI).

The experiment protocol consisted of performing cognitive evaluation and EEG microstates duration measurements before starting the TMS stimulations. After finishing a TMS sessions, cognitive evaluation was performed every 4 months approximately, and EEG microstates measurement were performed immediately before and after each TMS treatment session or TMS delivery, and also every 4 months after completion of the TMS stimulation sessions up to 2 years from the beginning of the clinical trial.

In the first 3 TMS stimulation sessions the subject received a different TMS protocol in each treatment session. The 3 TMS protocols were termed protocol 1, protocol 2, and protocol 3. When delivering TMS protocol 1, side effects of the TMS protocol was evaluated, to determine how tolerable is TMS protocol no. 1. If protocol 1 was intolerable, the TMS protocol was changed to TMS protocol no. 1-adjusted.

After the 3 TMS stimulation sessions, a physician or an expert monitoring the experiment, decided which protocol out of the TMS protocols used during the first 3 TMS stimulation sessions was the most suitable according to the microstates duration. In some embodiments, and in the experiment, a shorter duration of one or more microstates indicated a better outcome of the TMS protocol. This protocol was continued once a week for total of 16 weeks.

If during the treatment period the selected protocol demonstrated a reduction in shortening of the microstates duration, indicating lower efficiency, the protocol was replaced and the efficiency of the new protocol was further examined, for example until reaching a preferred and personalized TMS protocol for a specific patient.

The TMS protocols used in the experiment include:

### Protocol 1-TMS-EEG (1):

Stimulus location: about 4 centimeters anterior to a predetermined location of a primary motor cortex in a subject, for example above a prefrontal cortex.
Power (intensity): about 100% of a predetermined motor threshold in the subject, Frequency: about 10 Hz, Duration: about 2 sec, number of pulses: about 20,
Wait time: about 20 sec, number of trains: about 50.

### Protocol 2-TMS-EEG (2):

Stimulus location: about 5 centimeters posterior to a predetermined location of a primary motor cortex in a subject, for example above a parietal lobe or a precuneus brain region.
Power: about 100% of a predetermined motor threshold in the subject, Frequency: about 10 Hz, Duration: about 2 sec, number of pulses: about 20,
Wait time: about 20 sec, number of trains: about 50.

### Protocol 3-TMS-EEG (3):

Stimulus location: about 5 centimeters posterior to a predetermined location of a primary motor cortex in a subject, for example above a parietal lobe or a precuneus brain region.
Power: about 100% of a predetermined motor threshold in the subject, Frequency: about 18 Hz, Duration: about 2 sec, number of pulses: about 36,
Wait time: about 20 sec, number of trains: about 50.
   Protocol 1 adjusted-TMS-EEG (1 adj.):
Stimulus location: about 4 centimeters anterior to a predetermined location of a primary motor cortex in a subject, for example above a prefrontal cortex.
Power: will be determined individually as the maximal tolerable power for each subject,
Frequency: about 10 Hz, Duration: about 2 sec, number of pulses: about 20,
Wait time: about 20 sec, number of trains: about 50.

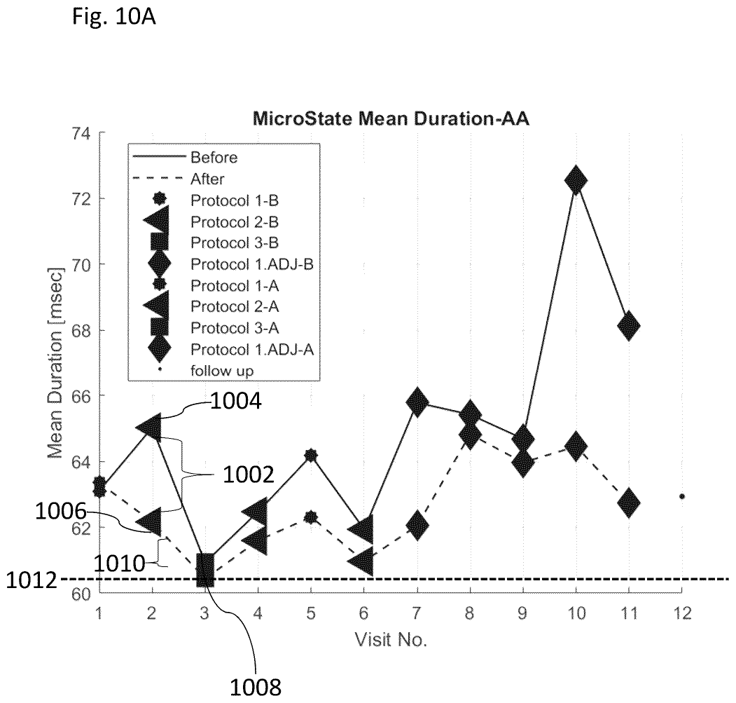

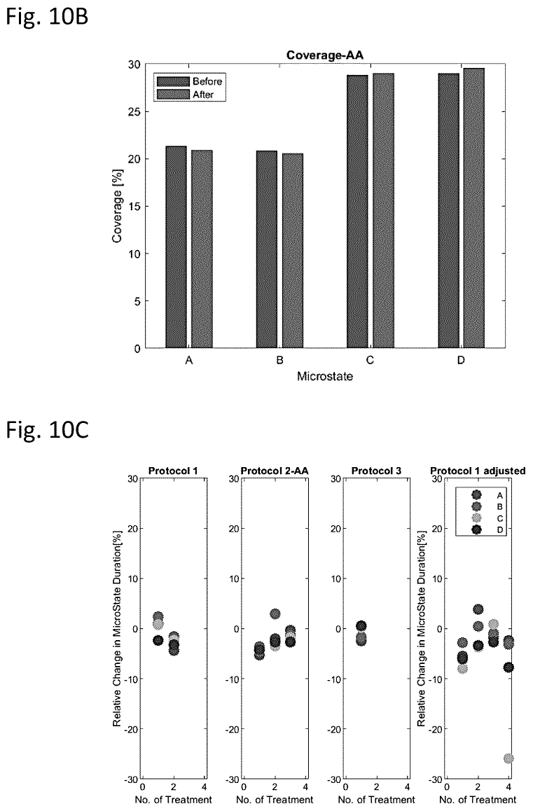

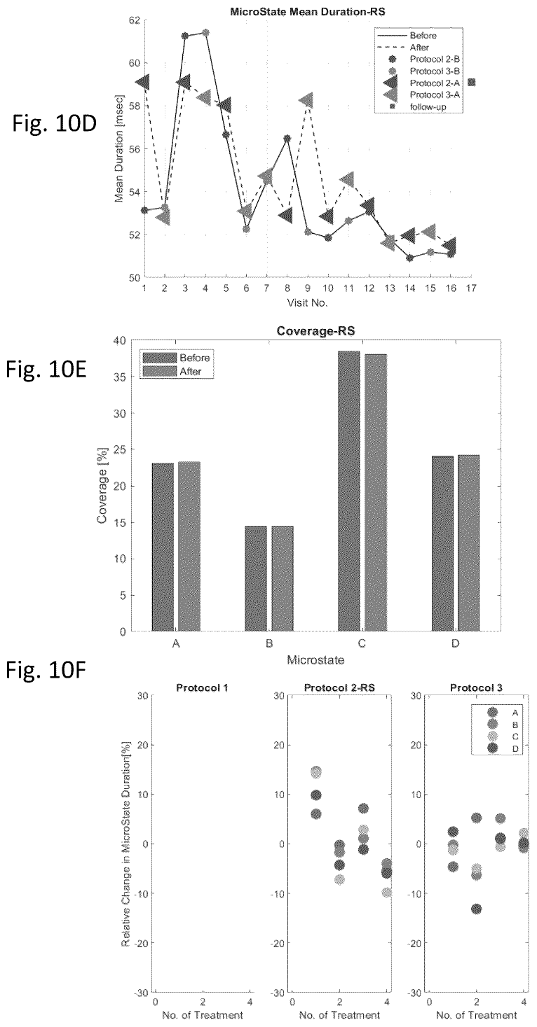

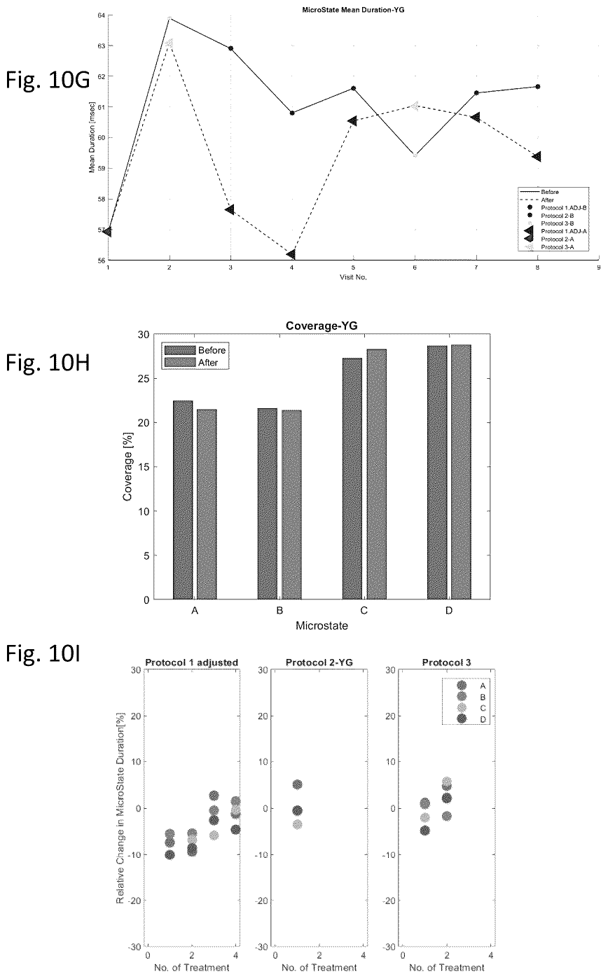

Reference is now made to FIG. 10A, depicting changes in microstates mean duration between different treatment sessions, as shown for a specific patient annotated as AA during the experiment.

According to some exemplary embodiments, in each visit, for example a treatment session, microstates mean duration was calculated before and after the delivery of TMS using a specific TMS protocol. In the experiment and in some embodiments of the invention, a change 1002 between the microstates mean duration 1004 before the TMS delivery and the microstates mean duration 1006 after the TMS delivery, indicate an immediate effect or a short-term effect of the treatment using a specific protocol by demonstrating a reduction in the microstates mean duration from approximately 65 msec to approximately 62 msec.

Monitoring the change in microstates mean duration before and after each treatment session allows, for example, to determine a short-term effect of each TMS protocol. For example, both protocols 1 and 3 had a much lower short-term effect, in comparison to protocol 2.

Alternatively or additionally, monitoring the change in microstates mean duration between treatment sessions allows, for example to determine an intermediate effect of a TMS protocol. For example, a comparison between microstates mean duration 1006 measured at an end of a treatment session, and microstates mean duration 1008 measured in the beginning of a following treatment session, reveals a further reduction 1010 in microstates mean duration, indicating a positive intermediate effect of the TMS protocol 2.

Alternatively or additionally, monitoring the change in microstates mean duration between treatment sessions allows, for example to estimate a brain responsiveness threshold and to optionally modify the treatment protocol accordingly. For example, monitoring the change in microstates mean duration between treatment sessions demonstrates that the lowest microstates mean duration is slightly above 60 msec. despite protocol changes. Identifying a lowest microstate mean duration after several treatment sessions may allow to estimate a brain responsiveness threshold 1012 at the lowest measured microstate mean duration. Estimating the brain responsiveness threshold 1012 may lead to performing further microstates measurement, optionally outside the clinic, to identify a preferred time window in which delivering a TMS to the patient will result with beneficial and cost-effective effect on the specific patient.

Additional analysis of microstates parameter was performed during the experiment and in some embodiments of the invention, for example to determine an efficacy of a TMS protocol and/or an efficacy of a TMS treatment. For example, as shown in FIG. 10B, a coverage percentage of each microstate is calculated before (left column) and after (right column) at least one treatment session. In another analysis example, a relative change in microstates duration is calculated for each microstate and for each treatment session, per a TMS protocol used in the treatment session (protocol 1, protocol 2, protocol 3 or protocol 1-adjusted).

FIGS. 10D-10I provide additional examples of similar analysis performed during the experiment for patients annotated as RS (FIGS. 10D-10F), and as YG (FIGS. 10G-10I).

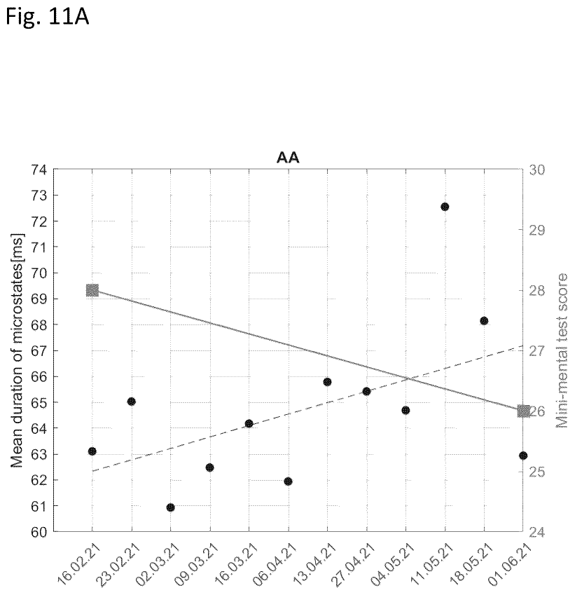

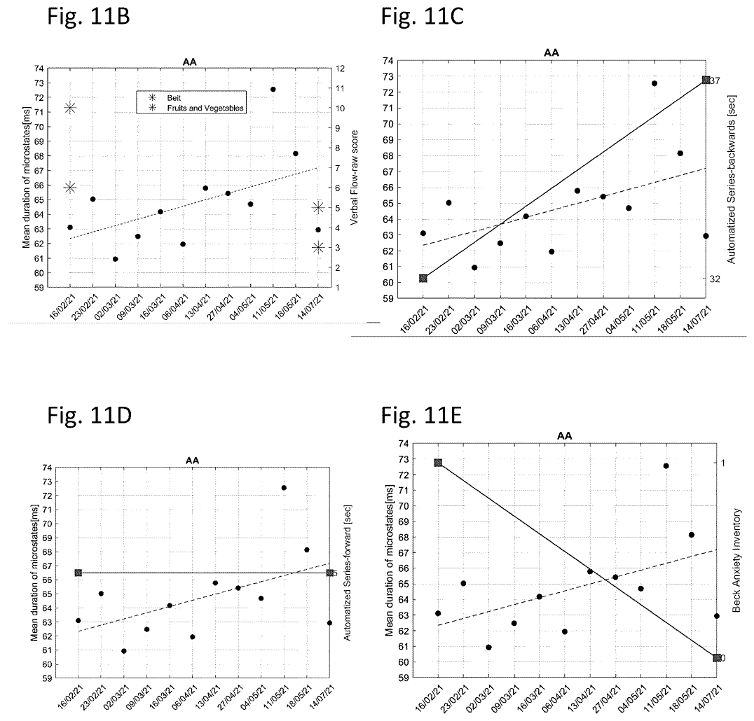

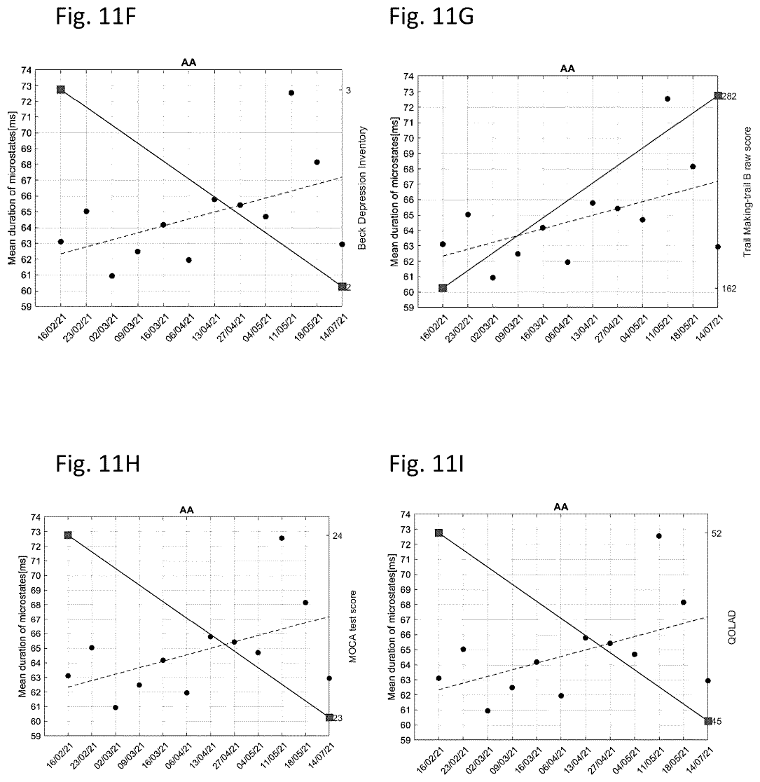

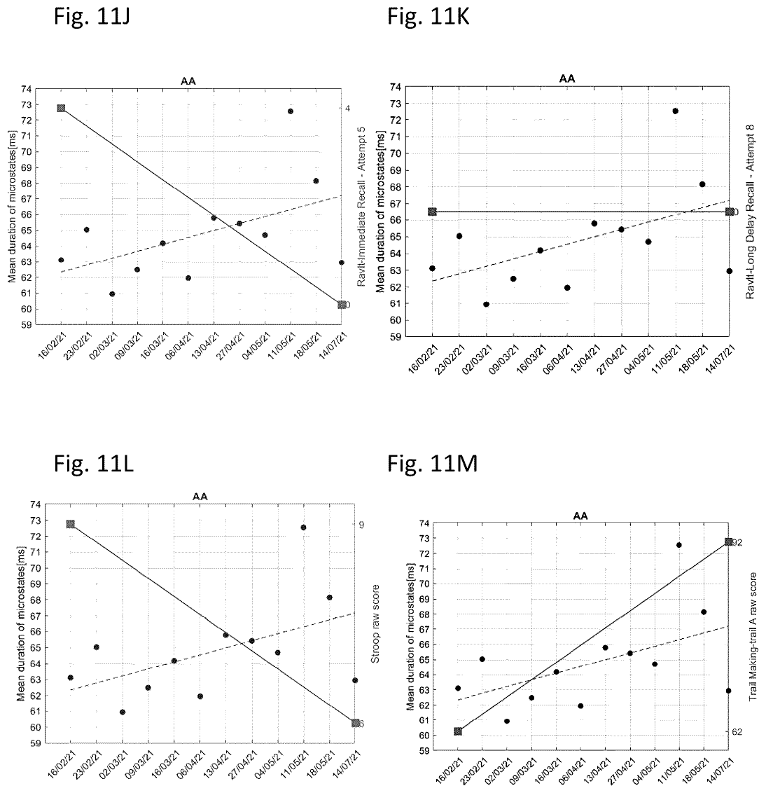

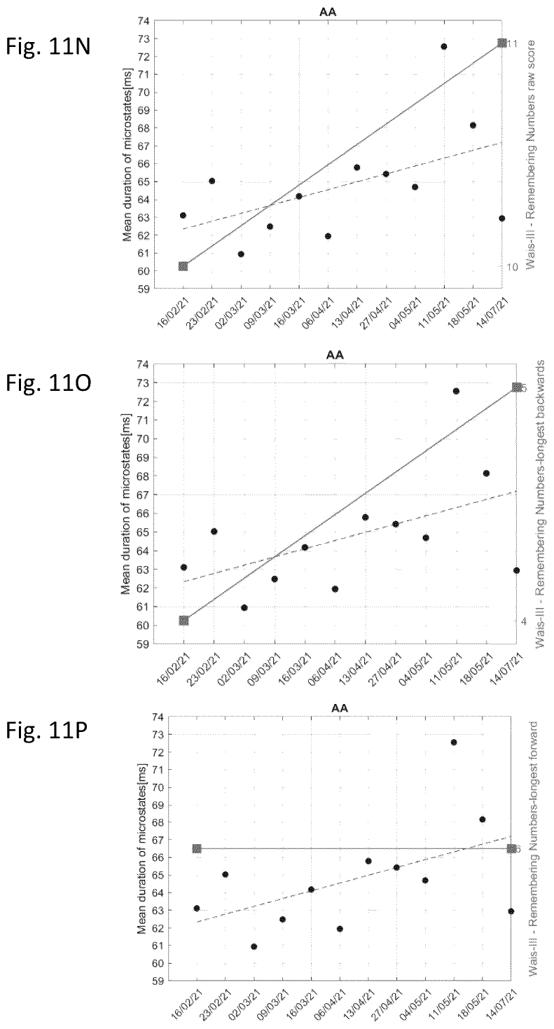

Reference is now made to FIG. 11A, depicting a correlation between changes in microstates mean duration and changes in mini-mental scores during different treatment sessions of a TMS treatment in a specific patient annotated as AA.

In the experiment and in some embodiments of the invention, one or more cognitive exams were performed, for example to identify a correlation between changes in overall mean duration of microstates and changes in a score of a cognitive exam during TMS treatment sessions. Monitoring a change in a score of cognitive exam may allow to identify the effect of a specific TMS protocol on a specific cognitive function domain tested by the cognitive exam. In addition, monitoring a change in a cognitive test during TMS treatment sessions may allow to identify when a cognitive state of a subject has reached a desired level, and to modify the TMS treatment plan accordingly, for example to stop or to delay TMS delivery or visits in the clinic if there is an improvement in the cognitive state of the subject.

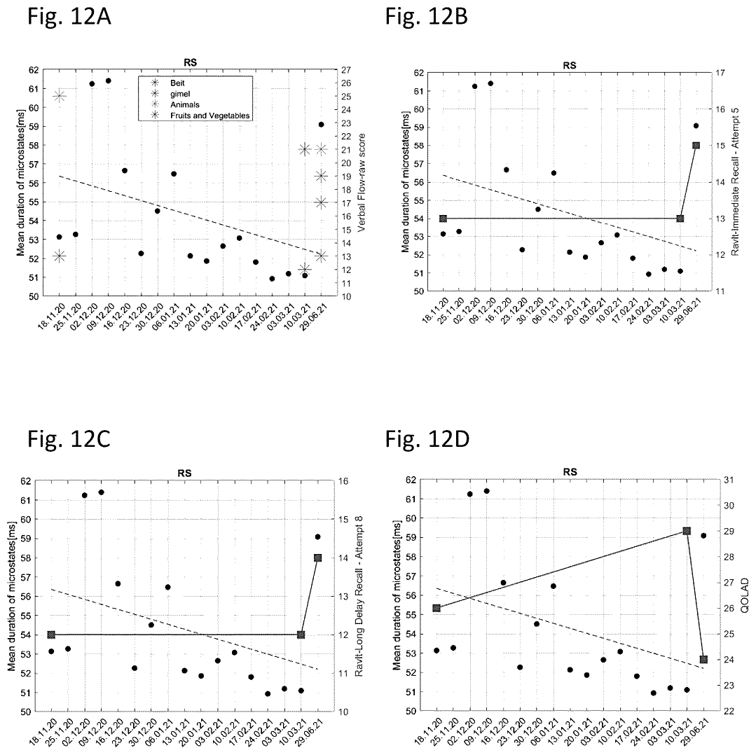

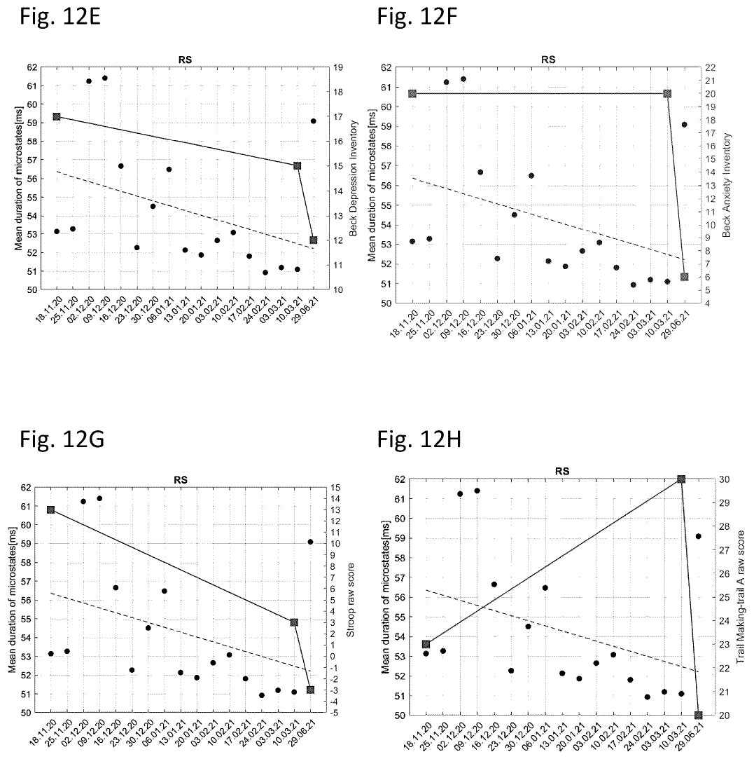

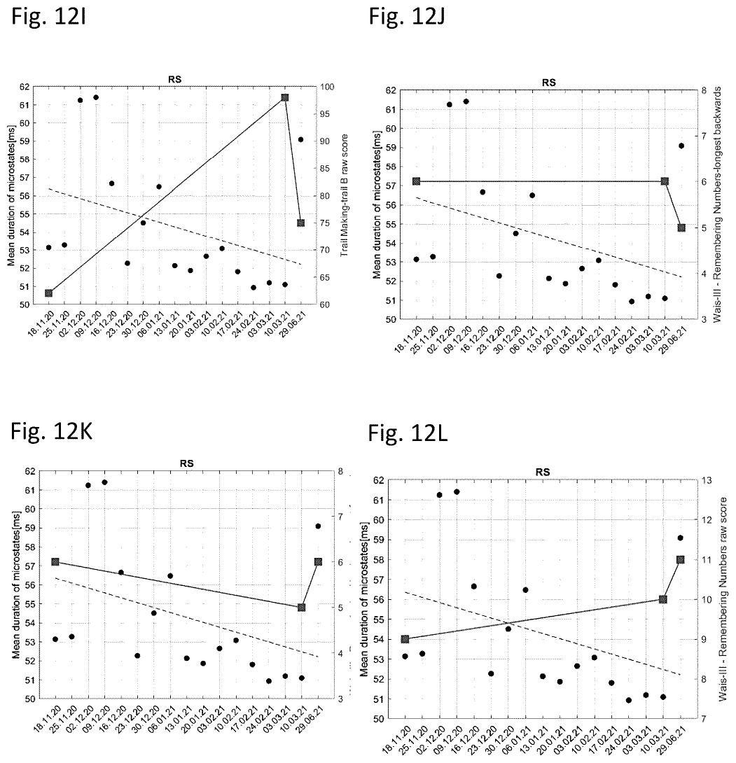

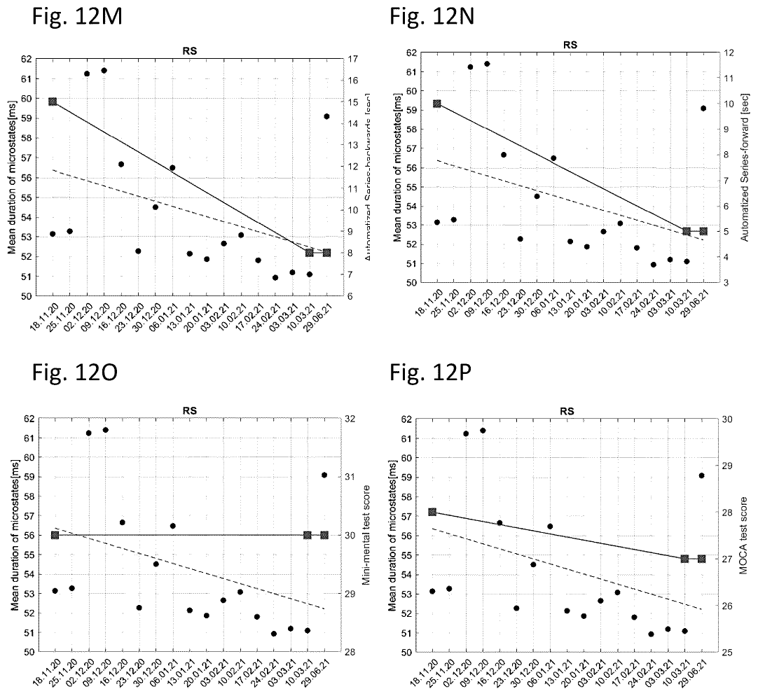

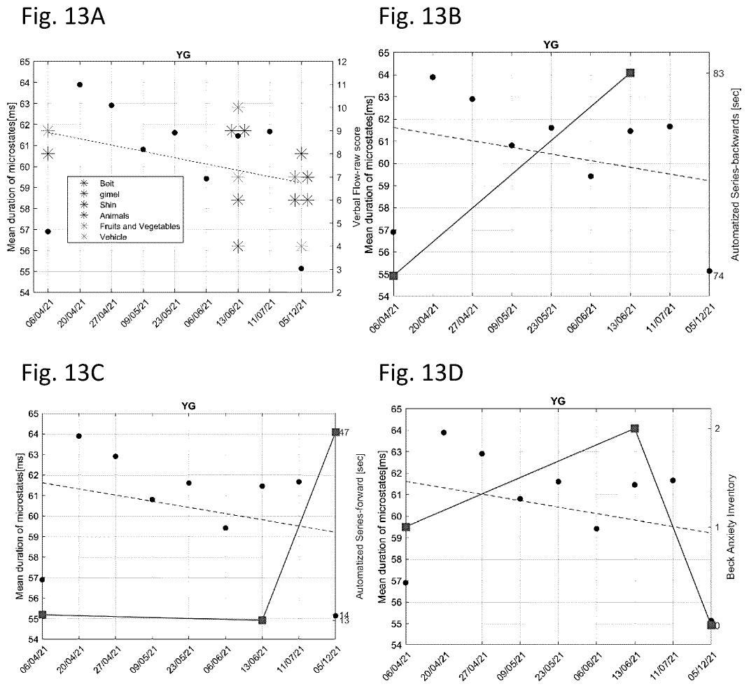

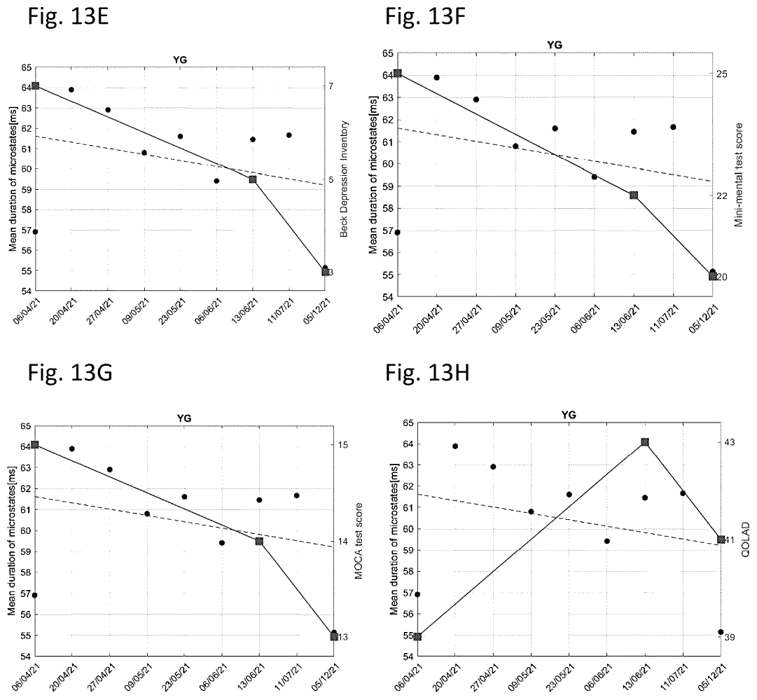

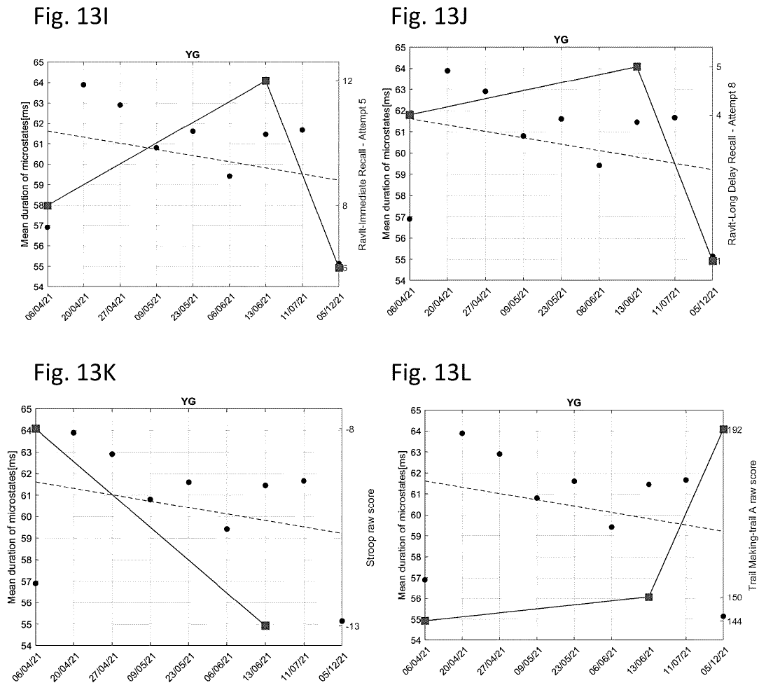

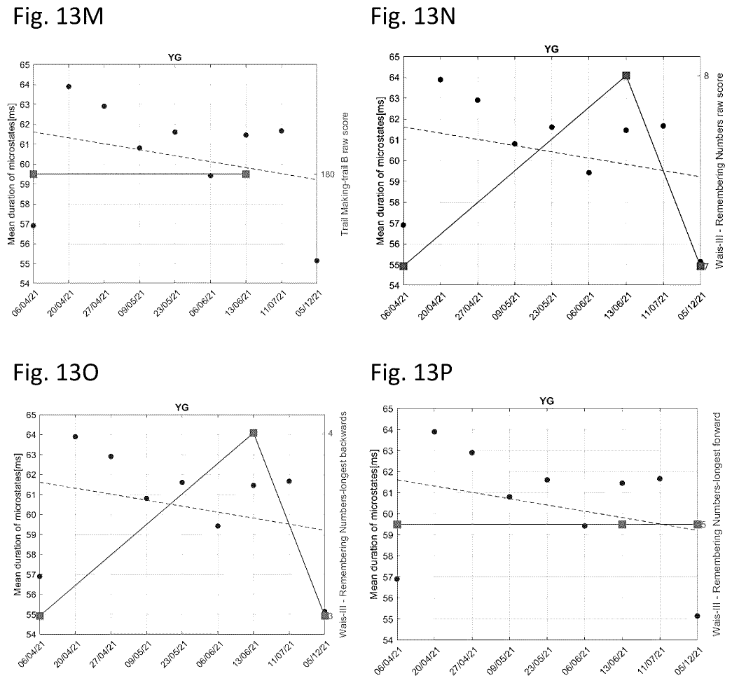

Additional examples for other cognitive exams and correlation between changes in microstates mean duration and changes in the cognitive exams scores during a TMS treatment are provided in FIGS. 11B-11P for patient AA, in FIGS. 12A-12P for patient RS, and in FIGS. 13A-13P for patient YG. In each graph the dashed line represents a trend of a change in the microstates mean duration and the solid line represents a trend of a change in the cognitive exam scores.

As used herein with reference to quantity or value, the term “about” means “within +10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-50. (canceled)

51. A system for determining an efficacy of a transcranial magnetic stimulation (TMS) treatment protocol, comprising:
a control circuitry, wherein the control circuitry is configured to:
analyzing EEG signals recorded during a TMS treatment delivered to a patient, to generate values of one or more EEG parameters;
determining an acute effect and/or an intermediate effect of the TMS protocol on a brain state of the patient based on a change in values of the one or more EEG parameters;
and
determining an efficacy of a TMS protocol used in the TMS treatment based on the determined acute effect and the determined intermediate effect.

52. The system of claim 51, wherein said intermediate effect of the TMS protocol is determined based on a change in microstates parameter values between values calculated using EEG signals recorded after completion of a treatment session of said TMS treatment and values calculated using EEG signals recorded during said treatment session.

53. The system of claim 51, wherein said control circuitry is further configured to determine a long-term effect of the TMS protocol, wherein said long-term effect is determined based on microstates measurements performed at least 24 hours following the TMS treatment.

54. A system according to claim 51, comprising:
a user interface operationally connected to the control circuitry, wherein the user interface is configured to generate a human detectable indication;
and wherein the control circuitry signals the user interface to generate the human detectable indication according to the determined efficacy.

55. A system according to claim 54, wherein the control circuitry signals the user interface to generate a human detectable indication with instructions to modify at least one parameter of the TMS protocol or to replace the TMS protocol with a different TMS protocol, if the determined efficacy is lower than a desired efficacy.

56. A system according to claim 55, wherein the control circuitry signals the user interface to generate a human detectable indication with instructions to stop the TMS treatment or to extend a length of an interval between two consecutive treatment sessions of the TMS treatment, if the determined efficacy is a desired efficacy or is larger than a desired efficacy.

57. A system according to claim 51, wherein the one or more EEG parameters comprise one or more EEG microstates parameters;
and wherein the control circuitry is configured to extract the one or more EEG microstates parameters from the EEG signals and to calculate values of the extracted one or more EEG parameters or central tendency thereof, during the analyzing, and to determine the acute effect and the intermediate effect based on a change in the calculated values of the extracted one or more EEG microstates parameters.

58. A system according to claim 57, wherein the one or more EEG microstates parameter comprise at least one of, duration of at least one EEG microstate, frequency of at least one EEG microstate, coverage of at least one microstate and transitions between two or more EEG microstates.

59. A system according to claim 51, further comprising:
at least one magnetic coil configured to be positioned close to, or in contact with a head of the patient;
at least one EEG electrode attached to a head of the patient, configured to record said EEG signals; and
a memory, wherein the memory stores at least one of said TMS treatment protocol,
wherein said control circuitry is configured to deliver TMS to the patient using the at least one TMS protocol in at least one treatment session.

60. A system according to claim 51, wherein the determined efficacy comprises determining that the TMS protocol has low efficacy in improving a cognitive state of the patient, based on the intermediate effect, and wherein the control circuitry is configured to modify the TMS treatment according to the determined efficacy.

61. A system according to claim 60, wherein the modifying comprises replacing the TMS protocol with a different TMS protocol, or modifying values of at least one parameter of the TMS protocol, based on the determined low efficacy.

62. A system according to claim 51, wherein the control circuitry is further configured to:
generate an indication to perform a cognitive examination of the patient using at least one cognitive exam, after completing the treatment session, to determine an intermediate effect of the at least one TMS treatment protocol, and
determine an efficacy of the TMS protocol based on the determined acute effect and the determined intermediate effect.

63. A system according to claim 51, wherein the control circuitry is configured to:
determine that a responsiveness of a brain of the patient to the delivered TMS is lower than a desired responsiveness level and/or that an effect of the TMS treatment has reached a maximal therapeutic effect after the treatment session, based on the determined efficacy; and
modify the TMS treatment and/or the TMS treatment protocol according to the determined responsiveness and/or the determined TMS treatment effect.

64. A system according to claim 63, wherein the modify the TMS treatment protocol by the control circuitry comprises replacing the TMS protocol with a different TMS protocol, or modifying values of at least one parameter of the TMS protocol.

65. A system according to claim 64, wherein the at least one parameter of the TMS protocol comprises at least one of, location of a magnetic coil on a head of the patient, intensity of a magnetic stimulation pulse, number of pulses per train of pulses, frequency of pulses during a train of pulses, and number of trains per treatment session.

66. A system according to claim 51, wherein modifying by the control circuitry comprises extending an interval between two consecutive treatment sessions of the TMS treatment.

67. A system according to claim 51, wherein modifying by the control circuitry comprises generating an indication by the control circuitry to administer at least one drug before and/or after delivery of the TMS.

68. A system according to claim 62, wherein said cognitive examination comprises at least one of, a mini-mental state exam (MMSE), a Montreal Cognitive Assessment (MoCA) and Rey's Auditory Verbal Learning Test (RAVLT) exam, Months backward test exam, Months forward test exam, Rey-Osterrieth Complex Figure Test (ROCFT) exam, Phonemic & Semantic Fluency exam, Digits span Forward & backwards exam, Stroop exam, Clock drawing exam, The Trail Making Test Part A and/or Part B, Functional Activities Questionnaire (FAQ), Quality of Life in Alzheimer's Disease (QOL-AD) Questionnaire, the Beck Depression Inventory (BDI, BDI-1A, BDI-II), the Beck Anxiety Inventory (BAI), and the Tablet-based Cognitive Assessment Tool (TabCAT).

69. A method for determining an efficacy of a transcranial magnetic stimulation (TMS) treatment, comprising:
delivering TMS to a patient using a TMS protocol in a TMS treatment session;
recording EEG signals before and after the delivering during the treatment session;
recording EEG signals after completion of the treatment session;
analyzing the recorded EEG signals to generate values of one or more EEG parameters;
determining an acute effect and an intermediate effect of the TMS protocol on a brain state of the patient based on a change in values of the one or more EEG parameters;
determining an efficacy of the TMS protocol based on the determined acute effect and the intermediate effect.

70. A method according to claim 69, wherein the one or more EEG parameters comprise one or more EEG microstates parameters, and wherein the analyzing comprises extracting the one or more EEG microstates parameters from the recorded EEG signals and measuring values of the one or more EEG microstates parameters or central tendency of the values, and wherein the acute effect and the intermediate effect are determined based on the measured values or the central tendency of the one or more EEG microstates parameters.

71. A method according to claim 70, wherein the measured one or more EEG microstates parameter comprise duration of at least one EEG microstate.

72. A method for determining an efficacy of a transcranial magnetic stimulation (TMS) treatment, comprising:
delivering TMS to a patient using a TMS protocol as part of a treatment session;
recording EEG signals before and after the delivering during the treatment session;
analyzing the recorded EEG signals to generate values of one or more EEG parameters;
determining an acute effect of the TMS protocol on a brain state of the patient based on values of the one or more EEG parameters;
performing a cognitive examination of the patient using at least one cognitive exam, after completing the treatment session to determine an intermediate effect of the TMS protocol, and
determining an efficacy of the TMS protocol based on the determined acute effect and the determined intermediate effect.

73. A method according to claim 72, wherein the at least one cognitive exam comprises at least one of, a mini-mental state exam (MMSE), a Montreal Cognitive Assessment (MoCA) and Rey's Auditory Verbal Learning Test (RAVLT) exam.
